SEQUENCE LISTING (19 sequences)

SEQ ID NO: 1 - DNA, 2982 bp, Artificial Sequence
  Synthetic Polynucleotide
atggcctcggcagctgtggagagcttcgtgaccaagcaactggacctgctggagctcgagagagacgcggaggtggaggagcgcaggtcctggcaggagaacatctctctgaaagagctccagagccgaggcgtgtgtttgctgaagctgcaggtatccagccagcgcactgggctgtacggacggctgctggtcacctttgagcccaggcgatacgggtccgcggcagctcttcccagtaacagctttacttctggtgatatcgtgggcctgtacgatgctgctaatgagggcagtcagctggccactgggatcttgacccgggtcacccagaagtcggtcacggtggcctttgatgagtcccacgatttccagttgagcttggaccgagagaattcctacagactgttaaaacttgccaatgatgtcacttacaggcgactgaaaaaagccctgattgctctaaagaagtatcattctggcccagcctcctcactcatagaagtgctctttggcagatctgctcccagtcctgccagtgaaatacacccgctgacattcttcaacacctgcctggacacctcccagaaagaagcggtttcatttgcgctgtctcagaaagaacttgccatcatccatggacctcctggcactgggaaaaccacgactgtggttgagatcattcttcaagctgtgaaacaaggcttaaaggttctgtgctgcgccccctccaacatcgccgtggacaatctggtggagcgcctggctctgtgtaagcagcggattctgcgcctgggacaccctgcccgcctcctggagtccattcagcagcactccctggatgcggttttagcgcggagcgacagtgcccagattgttgcagatatcaggaaggacatcgaccaggtctttgtgaaaaacaaaaagacccaggataagagagagaaaagtaattttcgaaatgaaattaagctgttaagaaaagaactgaaggagagggaagaagcagctatgctcgagagcctcacttcggcaaacgtggtccttgcaacaaacacaggtgcgtctgccgatggccccctgaagttgctgcccgagagctacttcgacgtggtggtcattgacgagtgtgcccaggccctcgaggcgagctgctggatccccctgctgaaggccagaaagtgcatcctggcgggcgatcacaagcagctgccccccaccacagtctctcacaaggctgcgctggcaggactgtcactcagcctgatggaacgcctggctgaggagtacggcgcgagggtggtgcggacactgacggtgcagtaccgcatgcaccaggctatcatgcgctgggcctcagacaccatgtaccttgggcagctcacagcccactcttccgtggcaaggcacctcctgagggacctcccaggtgtggctgccacagaagagacgggtgtgcccctgctcttggtggacaccgccggctgcgggctgtttgagctggaggaggaggacgaacagtcgaaagggaaccctggcgaagtccgcctcgtcagtttgcacatccaggctctggtggacgctggtgttccagcccgtgacattgctgtggtctcgccatacaacctccaggtggacctgctcagacagagccttgtgcacaggcaccctgagcttgaaatcaagtctgtcgatggcttccaaggccgagagaaggaggccgtgatactgtccttcgtcagatccaacaggaaaggtgaagttggttttcttgctgaggaccggaggatcaacgtggctgtcacccgtgcccgacgccacgtggcggtcatctgtgactcccgtactgtcaacaaccatgcatttttgaagaccctggtggagtatttcacacagcatggggaagtacgcacggcctttgagtatcttgacgatattgtcccagaaaactattcccatgagaactcccagggttccagccacgctgccaccaagccccagggacctgctgcgtccaccaggaccggaagccagcggcaggagggaggccaggaggctgcagcacctgccagacagggccggaagaagccggctgggaagtctctggcctctgaagctccatctcagcccagcctcaacggaggcagcccagagggagtggagagccaagatggcgtggaccacttccgggccatgatagtggagttcatggccagcaagaagatgcagttggagtttcctccttccctcaattcccacgacaggctgcgggtccaccaaatagccgaggagcacgggctgaggcacgacagttctggggaagggaagaggaggttcatcactgtgagcaagagggccccgcgaccccgagcagccctgggacccccagcagggaccggtggcccagcccctctccagccagtgccccctacccctgcgcagacagagcagcctcccagggagcagcgtggcccagaccagcctgatctgaggacgctgcacctggagagactgcagagggtcaggagcgcgcaggggcagcccgccagcaaggagcagcaggcctcagggcagcagaaacttccagaaaagaaaaagaaaaaagccaaaggacatccggccacagatctgcccacggaggaggactttgaggccctggtttctgccgccgttaaggctgataacacctgcggctttgccaagtgcacagccggcgtcacaaccctgggccagttctgccagctctgcagccgccgctactgcctcagccaccacctgcccgagatccatggctgcggtgagagggctcgcgcccatgcccggcagagaatcagccgggaaggggtcctctatgccggcagcgggaccaagaacggatccctggacccagccaagagggcccagctgcagaggaggctggataagaagctgagtgagctcagcaaccagaggaccagccggaggaaggagagggggacgtga

SEQ ID NO: 2 - protein, 993 aa, Homo Sapiens
MASAAVESFVTKQLDLLELERDAEVEERRSWQENISLKELQSRGVCLLKLQVSSQRTGLYGRLLVTFEPRRYGSAAALPSNSFTSGDIVGLYDAANEGSQLATGILTRVTQKSVTVAFDESHDFQLSLDRENSYRLLKLANDVTYRRLKKALIALKKYHSGPASSLIEVLFGRSAPSPASEIHPLTFFNTCLDTSQKEAVLFALSQKELAIIHGPPGTGKTTTVVEIILQAVKQGLKVLCCAPSNIAVDNLVERLALCKQRILRLGHPARLLESIQQHSLDAVLARSDSAQIVADIRKDIDQVFVKNKKTQDKREKSNFRNEIKLLRKELKEREEAAMLESLTSANVVLATNTGASADGPLKLLPESYFDVVVIDECAQALEASCWIPLLKARKCILAGDHKQLPPTTVSHKAALAGLSLSLMERLAEEYGARVVRTLTVQYRMHQAIMRWASDTMYLGQLTAHSSVARHLLRDLPGVAATEETGVPLLLVDTAGCGLFELEEEDEQSKGNPGEVRLVSLHIQALVDAGVPARDIAVVSPYNLQVDLLRQSLVHRHPELEIKSVDGFQGREKEAVILSFVRSNRKGEVGFLAEDRRINVAVTRARRHVAVICDSRTVNNHAFLKTLVEYFTQHGEVRTAFEYLDDIVPENYSHENSQGSSHAATKPQGPATSTRTGSQRQEGGQEAAAPARQGRKKPAGKSLASEAPSQPSLNGGSPEGVESQDGVDHFRAMIVEFMASKKMQLEFPPSLNSHDRLRVHQIAEEHGLRHDSSGEGKRRFITVSKRAPRPRAALGPPAGTGGPAPLQPVPPTPAQTEQPPREQRGPDQPDLRTLHLERLQRVRSAQGQPASKEQQASGQQKLPEKKKKKAKGHPATDLPTEEDFEALVSAAVKADNTCGFAKCTAGVTTLGQFCQLCSRRYCLSHHLPEIHGCGERARAHARQRISREGVLYAGSGTKNGSLDPAKRAQLQRRLDKKLSELSNQRTSRRKERGT

SEQ ID NO: 3 - DNA, 270 bp, Artificial Sequence
  Synthetic Polynucleotide
ccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggag

SEQ ID NO: 4 - DNA, 546 bp, Artificial Sequence
  Synthetic Polynucleotide
gaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaa

SEQ ID NO: 5 - DNA, 60 bp, Artificial Sequence
  Synthetic Polynucleotide
gtaagtttagtctttttgtcttttatttcaggtcccggatccggtggtggtgcaaatcaa

SEQ ID NO: 6 - DNA, 810 bp, Artificial Sequence
  Synthetic Polynucleotide
atgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 7 - DNA, 6838 bp, Artificial Sequence
  Synthetic Polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggttttaagggccgaggcggccagatctttcgaagatatcggcgccgctagcgcgcgtttaaaccctgcaggtctagaatggcctcggcagctgtggagagcttcgtgaccaagcaactggacctgctggagctcgagagagacgcggaggtggaggagcgcaggtcctggcaggagaacatctctctgaaagagctccagagccgaggcgtgtgtttgctgaagctgcaggtatccagccagcgcactgggctgtacggacggctgctggtcacctttgagcccaggcgatacgggtccgcggcagctcttcccagtaacagctttacttctggtgatatcgtgggcctgtacgatgctgctaatgagggcagtcagctggccactgggatcttgacccgggtcacccagaagtcggtcacggtggcctttgatgagtcccacgatttccagttgagcttggaccgagagaattcctacagactgttaaaacttgccaatgatgtcacttacaggcgactgaaaaaagccctgattgctctaaagaagtatcattctggcccagcctcctcactcatagaagtgctctttggcagatctgctcccagtcctgccagtgaaatacacccgctgacattcttcaacacctgcctggacacctcccagaaagaagcggtttcatttgcgctgtctcagaaagaacttgccatcatccatggacctcctggcactgggaaaaccacgactgtggttgagatcattcttcaagctgtgaaacaaggcttaaaggttctgtgctgcgccccctccaacatcgccgtggacaatctggtggagcgcctggctctgtgtaagcagcggattctgcgcctgggacaccctgcccgcctcctggagtccattcagcagcactccctggatgcggttttagcgcggagcgacagtgcccagattgttgcagatatcaggaaggacatcgaccaggtctttgtgaaaaacaaaaagacccaggataagagagagaaaagtaattttcgaaatgaaattaagctgttaagaaaagaactgaaggagagggaagaagcagctatgctcgagagcctcacttcggcaaacgtggtccttgcaacaaacacaggtgcgtctgccgatggccccctgaagttgctgcccgagagctacttcgacgtggtggtcattgacgagtgtgcccaggccctcgaggcgagctgctggatccccctgctgaaggccagaaagtgcatcctggcgggcgatcacaagcagctgccccccaccacagtctctcacaaggctgcgctggcaggactgtcactcagcctgatggaacgcctggctgaggagtacggcgcgagggtggtgcggacactgacggtgcagtaccgcatgcaccaggctatcatgcgctgggcctcagacaccatgtaccttgggcagctcacagcccactcttccgtggcaaggcacctcctgagggacctcccaggtgtggctgccacagaagagacgggtgtgcccctgctcttggtggacaccgccggctgcgggctgtttgagctggaggaggaggacgaacagtcgaaagggaaccctggcgaagtccgcctcgtcagtttgcacatccaggctctggtggacgctggtgttccagcccgtgacattgctgtggtctcgccatacaacctccaggtggacctgctcagacagagccttgtgcacaggcaccctgagcttgaaatcaagtctgtcgatggcttccaaggccgagagaaggaggccgtgatactgtccttcgtcagatccaacaggaaaggtgaagttggttttcttgctgaggaccggaggatcaacgtggctgtcacccgtgcccgacgccacgtggcggtcatctgtgactcccgtactgtcaacaaccatgcatttttgaagaccctggtggagtatttcacacagcatggggaagtacgcacggcctttgagtatcttgacgatattgtcccagaaaactattcccatgagaactcccagggttccagccacgctgccaccaagccccagggacctgctgcgtccaccaggaccggaagccagcggcaggagggaggccaggaggctgcagcacctgccagacagggccggaagaagccggctgggaagtctctggcctctgaagctccatctcagcccagcctcaacggaggcagcccagagggagtggagagccaagatggcgtggaccacttccgggccatgatagtggagttcatggccagcaagaagatgcagttggagtttcctccttccctcaattcccacgacaggctgcgggtccaccaaatagccgaggagcacgggctgaggcacgacagttctggggaagggaagaggaggttcatcactgtgagcaagagggccccgcgaccccgagcagccctgggacccccagcagggaccggtggcccagcccctctccagccagtgccccctacccctgcgcagacagagcagcctcccagggagcagcgtggcccagaccagcctgatctgaggacgctgcacctggagagactgcagagggtcaggagcgcgcaggggcagcccgccagcaaggagcagcaggcctcagggcagcagaaacttccagaaaagaaaaagaaaaaagccaaaggacatccggccacagatctgcccacggaggaggactttgaggccctggtttctgccgccgttaaggctgataacacctgcggctttgccaagtgcacagccggcgtcacaaccctgggccagttctgccagctctgcagccgccgctactgcctcagccaccacctgcccgagatccatggctgcggtgagagggctcgcgcccatgcccggcagagaatcagccgggaaggggtcctctatgccggcagcgggaccaagaacggatccctggacccagccaagagggcccagctgcagaggaggctggataagaagctgagtgagctcagcaaccagaggaccagccggaggaaggagagggggacgtgaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 8 - DNA, 6816 bp, Artificial Sequence
  Synthetic Polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtaccgaattcacgcgtgaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaacgcgtcgaagggcgaattctgcagataactggtaagtttagtcttttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcttaagggccgaggcggccagatctttcgaagatatcggcgccgctagcgcgcgtttaaaccctgcaggtctagaatggcctcggcagctgtggagagcttcgtgaccaagcaactggacctgctggagctcgagagagacgcggaggtggaggagcgcaggtcctggcaggagaacatctctctgaaagagctccagagccgaggcgtgtgtttgctgaagctgcaggtatccagccagcgcactgggctgtacggacggctgctggtcacctttgagcccaggcgatacgggtccgcggcagctcttcccagtaacagctttacttctggtgatatcgtgggcctgtacgatgctgctaatgagggcagtcagctggccactgggatcttgacccgggtcacccagaagtcggtcacggtggcctttgatgagtcccacgatttccagttgagcttggaccgagagaattcctacagactgttaaaacttgccaatgatgtcacttacaggcgactgaaaaaagccctgattgctctaaagaagtatcattctggcccagcctcctcactcatagaagtgctctttggcagatctgctcccagtcctgccagtgaaatacacccgctgacattcttcaacacctgcctggacacctcccagaaagaagcggtttcatttgcgctgtctcagaaagaacttgccatcatccatggacctcctggcactgggaaaaccacgactgtggttgagatcattcttcaagctgtgaaacaaggcttaaaggttctgtgctgcgccccctccaacatcgccgtggacaatctggtggagcgcctggctctgtgtaagcagcggattctgcgcctgggacaccctgcccgcctcctggagtccattcagcagcactccctggatgcggttttagcgcggagcgacagtgcccagattgttgcagatatcaggaaggacatcgaccaggtctttgtgaaaaacaaaaagacccaggataagagagagaaaagtaattttcgaaatgaaattaagctgttaagaaaagaactgaaggagagggaagaagcagctatgctcgagagcctcacttcggcaaacgtggtccttgcaacaaacacaggtgcgtctgccgatggccccctgaagttgctgcccgagagctacttcgacgtggtggtcattgacgagtgtgcccaggccctcgaggcgagctgctggatccccctgctgaaggccagaaagtgcatcctggcgggcgatcacaagcagctgccccccaccacagtctctcacaaggctgcgctggcaggactgtcactcagcctgatggaacgcctggctgaggagtacggcgcgagggtggtgcggacactgacggtgcagtaccgcatgcaccaggctatcatgcgctgggcctcagacaccatgtaccttgggcagctcacagcccactcttccgtggcaaggcacctcctgagggacctcccaggtgtggctgccacagaagagacgggtgtgcccctgctcttggtggacaccgccggctgcgggctgtttgagctggaggaggaggacgaacagtcgaaagggaaccctggcgaagtccgcctcgtcagtttgcacatccaggctctggtggacgctggtgttccagcccgtgacattgctgtggtctcgccatacaacctccaggtggacctgctcagacagagccttgtgcacaggcaccctgagcttgaaatcaagtctgtcgatggcttccaaggccgagagaaggaggccgtgatactgtccttcgtcagatccaacaggaaaggtgaagttggttttcttgctgaggaccggaggatcaacgtggctgtcacccgtgcccgacgccacgtggcggtcatctgtgactcccgtactgtcaacaaccatgcatttttgaagaccctggtggagtatttcacacagcatggggaagtacgcacggcctttgagtatcttgacgatattgtcccagaaaactattcccatgagaactcccagggttccagccacgctgccaccaagccccagggacctgctgcgtccaccaggaccggaagccagcggcaggagggaggccaggaggctgcagcacctgccagacagggccggaagaagccggctgggaagtctctggcctctgaagctccatctcagcccagcctcaacggaggcagcccagagggagtggagagccaagatggcgtggaccacttccgggccatgatagtggagttcatggccagcaagaagatgcagttggagtttcctccttccctcaattcccacgacaggctgcgggtccaccaaatagccgaggagcacgggctgaggcacgacagttctggggaagggaagaggaggttcatcactgtgagcaagagggccccgcgaccccgagcagccctgggacccccagcagggaccggtggcccagcccctctccagccagtgccccctacccctgcgcagacagagcagcctcccagggagcagcgtggcccagaccagcctgatctgaggacgctgcacctggagagactgcagagggtcaggagcgcgcaggggcagcccgccagcaaggagcagcaggcctcagggcagcagaaacttccagaaaagaaaaagaaaaaagccaaaggacatccggccacagatctgcccacggaggaggactttgaggccctggtttctgccgccgttaaggctgataacacctgcggctttgccaagtgcacagccggcgtcacaaccctgggccagttctgccagctctgcagccgccgctactgcctcagccaccacctgcccgagatccatggctgcggtgagagggctcgcgcccatgcccggcagagaatcagccgggaaggggtcctctatgccggcagcgggaccaagaacggatccctggacccagccaagagggcccagctgcagaggaggctggataagaagctgagtgagctcagcaaccagaggaccagccggaggaaggagagggggacgtgaaagcttgatatcctcgaggctagcggatccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 9 - DNA, 6838 bp, Artificial Sequence
  Synthetic Polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggttttaagggccgaggcggccagatctttcgaagatatcggcgccgctagcgcgcgtttaaaccctgcaggtctagaatggcctcggcagctgtggagagcttcgtgaccaagcaactggacctgctggagctcgagagagacgcggaggtggaggagcgcaggtcctggcaggagaacatctctctgaaagagctccagagccgaggcgtgtgtttgctgaagctgcaggtatccagccagcgcactgggctgtacggacggctgctggtcacctttgagcccaggcgatacgggtccgcggcagctcttcccagtaacagctttacttctggtgatatcgtgggcctgtacgatgctgctaatgagggcagtcagctggccactgggatcttgacccgggtcacccagaagtcggtcacggtggcctttgatgagtcccacgatttccagttgagcttggaccgagagaattcctacagactgttaaaacttgccaatgatgtcacttacaggcgactgaaaaaagccctgattgctctaaagaagtatcattctggcccagcctcctcactcatagaagtgctctttggcagatctgctcccagtcctgccagtgaaatacacccgctgacattcttcaacacctgcctggacacctcccagaaagaagcggtttcatttgcgctgtctcagaaagaacttgccatcatccatggacctcctggcactgggaaaaccacgactgtggttgagatcattcttcaagctgtgaaacaaggcttaaaggttctgtgctgcgccccctccaacatcgccgtggacaatctggtggagcgcctggctctgtgtaagcagcggattctgcgcctgggacaccctgcccgcctcctggagtccattcagcagcactccctggatgcggttttagcgcggagcgacagtgcccagattgttgcagatatcaggaaggacatcgaccaggtctttgtgaaaaacaaaaagacccaggataagagagagaaaagtaattttcgaaatgaaattaagctgttaagaaaagaactgaaggagagggaagaagcagctatgctcgagagcctcacttcggcaaacgtggtccttgcaacaaacacaggtgcgtctgccgatggccccctgaagttgctgcccgagagctacttcgacgtggtggtcattgacgagtgtgcccaggccctcgaggcgagctgctggatccccctgctgaaggccagaaagtgcatcctggcgggcgatcacaagcagctgccccccaccacagtctctcacaaggctgcgctggcaggactgtcactcagcctgatggaacgcctggctgaggagtacggcgcgagggtggtgcggacactgacggtgcagtaccgcatgcaccaggctatcatgcgctgggcctcagacaccatgtaccttgggcagctcacagcccactcttccgtggcaaggcacctcctgagggacctcccaggtgtggctgccacagaagagacgggtgtgcccctgctcttggtggacaccgccggctgcgggctgtttgagctggaggaggaggacgaacagtcgaaagggaaccctggcgaagtccgcctcgtcagtttgcacatccaggctctggtggacgctggtgttccagcccgtgacattgctgtggtctcgccatacaacctccaggtggacctgctcagacagagccttgtgcacaggcaccctgagcttgaaatcaagtctgtcgatggcttccaaggccgagagaaggaggccgtgatactgtccttcgtcagatccaacaggaaaggtgaagttggttttcttgctgaggaccggaggatcaacgtggctgtcacccgtgcccgacgccacgtggcggtcatctgtgactcccgtactgtcaacaaccatgcatttttgaagaccctggtggagtatttcacacagcatggggaagtacgcacggcctttgagtatcttgacgatattgtcccagaaaactattcccatgagaactcccagggttccagccacgctgccaccaagccccagggacctgctgcgtccaccaggaccggaagccagcggcaggagggaggccaggaggctgcagcacctgccagacagggccggaagaagccggctgggaagtctctggcctctgaagctccatctcagcccagcctcaacggaggcagcccagagggagtggagagccaagatggcgtggaccacttccgggccatgatagtggagttcatggccagcaagaagatgcagttggagtttcctccttccctcaattcccacgacaggctgcgggtccaccaaatagccgaggagcacgggctgaggcacgacagttctggggaagggaagaggaggttcatcactgtgagcaagagggccccgcgaccccgagcagccctgggacccccagcagggaccggtggcccagcccctctccagccagtgccccctacccctgcgcagacagagcagcctcccagggagcagcgtggcccagaccagcctgatctgaggacgctgcacctggagagactgcagagggtcaggagcgcgcaggggcagcccgccagcaaggagcagcaggcctcagggcagcagaaacttccagaaaagaaaaagaaaaaagccaaaggacatccggccacagatctgcccacggaggaggactttgaggccctggtttctgccgccgttaaggctgataacacctgcggctttgccaagtgcacagccggcgtcacaaccctgggccagttctgccagctctgcagccgccgctactgcctcagccaccacctgcccgagatccatggctgcggtgagagggctcgcgcccatgcccggcagagaatcagccgggaaggggtcctctatgccggcagcgggaccaagaacggatccctggacccagccaagagggcccagctgcagaggaggctggataagaagctgagtgagctcagcaaccagaggaccagccggaggaaggagagggggacgtgaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 10 - DNA, 6816 bp, Artificial Sequence
  Synthetic Polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtaccgaattcacgcgtgaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaacgcgtcgaagggcgaattctgcagataactggtaagtttagtcttttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcttaagggccgaggcggccagatctttcgaagatatcggcgccgctagcgcgcgtttaaaccctgcaggtctagaatggcctcggcagctgtggagagcttcgtgaccaagcaactggacctgctggagctcgagagagacgcggaggtggaggagcgcaggtcctggcaggagaacatctctctgaaagagctccagagccgaggcgtgtgtttgctgaagctgcaggtatccagccagcgcactgggctgtacggacggctgctggtcacctttgagcccaggcgatacgggtccgcggcagctcttcccagtaacagctttacttctggtgatatcgtgggcctgtacgatgctgctaatgagggcagtcagctggccactgggatcttgacccgggtcacccagaagtcggtcacggtggcctttgatgagtcccacgatttccagttgagcttggaccgagagaattcctacagactgttaaaacttgccaatgatgtcacttacaggcgactgaaaaaagccctgattgctctaaagaagtatcattctggcccagcctcctcactcatagaagtgctctttggcagatctgctcccagtcctgccagtgaaatacacccgctgacattcttcaacacctgcctggacacctcccagaaagaagcggtttcatttgcgctgtctcagaaagaacttgccatcatccatggacctcctggcactgggaaaaccacgactgtggttgagatcattcttcaagctgtgaaacaaggcttaaaggttctgtgctgcgccccctccaacatcgccgtggacaatctggtggagcgcctggctctgtgtaagcagcggattctgcgcctgggacaccctgcccgcctcctggagtccattcagcagcactccctggatgcggttttagcgcggagcgacagtgcccagattgttgcagatatcaggaaggacatcgaccaggtctttgtgaaaaacaaaaagacccaggataagagagagaaaagtaattttcgaaatgaaattaagctgttaagaaaagaactgaaggagagggaagaagcagctatgctcgagagcctcacttcggcaaacgtggtccttgcaacaaacacaggtgcgtctgccgatggccccctgaagttgctgcccgagagctacttcgacgtggtggtcattgacgagtgtgcccaggccctcgaggcgagctgctggatccccctgctgaaggccagaaagtgcatcctggcgggcgatcacaagcagctgccccccaccacagtctctcacaaggctgcgctggcaggactgtcactcagcctgatggaacgcctggctgaggagtacggcgcgagggtggtgcggacactgacggtgcagtaccgcatgcaccaggctatcatgcgctgggcctcagacaccatgtaccttgggcagctcacagcccactcttccgtggcaaggcacctcctgagggacctcccaggtgtggctgccacagaagagacgggtgtgcccctgctcttggtggacaccgccggctgcgggctgtttgagctggaggaggaggacgaacagtcgaaagggaaccctggcgaagtccgcctcgtcagtttgcacatccaggctctggtggacgctggtgttccagcccgtgacattgctgtggtctcgccatacaacctccaggtggacctgctcagacagagccttgtgcacaggcaccctgagcttgaaatcaagtctgtcgatggcttccaaggccgagagaaggaggccgtgatactgtccttcgtcagatccaacaggaaaggtgaagttggttttcttgctgaggaccggaggatcaacgtggctgtcacccgtgcccgacgccacgtggcggtcatctgtgactcccgtactgtcaacaaccatgcatttttgaagaccctggtggagtatttcacacagcatggggaagtacgcacggcctttgagtatcttgacgatattgtcccagaaaactattcccatgagaactcccagggttccagccacgctgccaccaagccccagggacctgctgcgtccaccaggaccggaagccagcggcaggagggaggccaggaggctgcagcacctgccagacagggccggaagaagccggctgggaagtctctggcctctgaagctccatctcagcccagcctcaacggaggcagcccagagggagtggagagccaagatggcgtggaccacttccgggccatgatagtggagttcatggccagcaagaagatgcagttggagtttcctccttccctcaattcccacgacaggctgcgggtccaccaaatagccgaggagcacgggctgaggcacgacagttctggggaagggaagaggaggttcatcactgtgagcaagagggccccgcgaccccgagcagccctgggacccccagcagggaccggtggcccagcccctctccagccagtgccccctacccctgcgcagacagagcagcctcccagggagcagcgtggcccagaccagcctgatctgaggacgctgcacctggagagactgcagagggtcaggagcgcgcaggggcagcccgccagcaaggagcagcaggcctcagggcagcagaaacttccagaaaagaaaaagaaaaaagccaaaggacatccggccacagatctgcccacggaggaggactttgaggccctggtttctgccgccgttaaggctgataacacctgcggctttgccaagtgcacagccggcgtcacaaccctgggccagttctgccagctctgcagccgccgctactgcctcagccaccacctgcccgagatccatggctgcggtgagagggctcgcgcccatgcccggcagagaatcagccgggaaggggtcctctatgccggcagcgggaccaagaacggatccctggacccagccaagagggcccagctgcagaggaggctggataagaagctgagtgagctcagcaaccagaggaccagccggaggaaggagagggggacgtgaaagcttgatatcctcgaggctagcggatccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 11 - DNA, 141 bp, Artificial Sequence
  Synthetic Polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 12 - DNA, 141 bp, Artificial Sequence
  Synthetic Polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 13 - DNA, 668 bp, Artificial Sequence
  Synthetic Polynucleotide
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctca

SEQ ID NO: 14 - DNA, 280 bp, Artificial Sequence
  Synthetic Polynucleotide
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctac

SEQ ID NO: 15 - DNA, 98 bp, Artificial Sequence
  Synthetic Polynucloetide
gtaagtttagtcttttttgtcttttatttcaggtcccggatccggtggtggtgcaaatcaaagaactgctcctcagtggatgttgcctttacttctag

SEQ ID NO: 16 - DNA, 147 bp, Artificial Sequence
  Synthetic Polynucleotide
cctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattc

SEQ ID NO: 17 - DNA, 6805 bp, Artificial Sequence
  Synthetic Polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgacggatccgctagcctcgaggatatcaagctttcacgtccccctctccttcctccggctggtcctctggttgctgagctcactcagcttcttatccagcctcctctgcagctgggccctcttggctgggtccagggatccgttcttggtcccgctgccggcatagaggaccccttcccggctgattctctgccgggcatgggcgcgagccctctcaccgcagccatggatctcgggcaggtggtggctgaggcagtagcggcggctgcagagctggcagaactggcccagggttgtgacgccggctgtgcacttggcaaagccgcaggtgttatcagccttaacggcggcagaaaccagggcctcaaagtcctcctccgtgggcagatctgtggccggatgtcctttggcttttttctttttcttttctggaagtttctgctgccctgaggcctgctgctccttgctggcgggctgcccctgcgcgctcctgaccctctgcagtctctccaggtgcagcgtcctcagatcaggctggtctgggccacgctgctccctgggaggctgctctgtctgcgcaggggtagggggcactggctggagaggggctgggccaccggtccctgctgggggtcccagggctgctcggggtcgcggggccctcttgctcacagtgatgaacctcctcttcccttccccagaactgtcgtgcctcagcccgtgctcctcggctatttggtggacccgcagcctgtcgtgggaattgagggaaggaggaaactccaactgcatcttcttgctggccatgaactccactatcatggcccggaagtggtccacgccatcttggctctccactccctctgggctgcctccgttgaggctgggctgagatggagcttcagaggccagagacttcccagccggcttcttccggccctgtctggcaggtgctgcagcctcctggcctccctcctgccgctggcttccggtcctggtggacgcagcaggtccctggggcttggtggcagcgtggctggaaccctgggagttctcatgggaatagttttctgggacaatatcgtcaagatactcaaaggccgtgcgtacttccccatgctgtgtgaaatactccaccagggtcttcaaaaatgcatggttgttgacagtacgggagtcacagatgaccgccacgtggcgtcgggcacgggtgacagccacgttgatcctccggtcctcagcaagaaaaccaacttcacctttcctgttggatctgacgaaggacagtatcacggcctccttctctcggccttggaagccatcgacagacttgatttcaagctcagggtgcctgtgcacaaggctctgtctgagcaggtccacctggaggttgtatggcgagaccacagcaatgtcacgggctggaacaccagcgtccaccagagcctggatgtgcaaactgacgaggcggacttcgccagggttccctttcgactgttcgtcctcctcctccagctcaaacagcccgcagccggcggtgtccaccaagagcaggggcacacccgtctcttctgtggcagccacacctgggaggtccctcaggaggtgccttgccacggaagagtgggctgtgagctgcccaaggtacatggtgtctgaggcccagcgcatgatagcctggtgcatgcggtactgcaccgtcagtgtccgcaccaccctcgcgccgtactcctcagccaggcgttccatcaggctgagtgacagtcctgccagcgcagccttgtgagagactgtggtggggggcagctgcttgtgatcgcccgccaggatgcactttctggccttcagcagggggatccagcagctcgcctcgagggcctgggcacactcgtcaatgaccaccacgtcgaagtagctctcgggcagcaacttcagggggccatcggcagacgcacctgtgtttgttgcaaggaccacgtttgccgaagtgaggctctcgagcatagctgcttcttccctctccttcagttcttttcttaacagcttaatttcatttcgaaaattacttttctctctcttatcctgggtctttttgtttttcacaaagacctggtcgatgtccttcctgatatctgcaacaatctgggcactgtcgctccgcgctaaaaccgcatccagggagtgctgctgaatggactccaggaggcgggcagggtgtcccaggcgcagaatccgctgcttacacagagccaggcgctccaccagattgtccacggcgatgttggagggggcgcagcacagaacctttaagccttgtttcacagcttgaagaatgatctcaaccacagtcgtggttttcccagtgccaggaggtccatggatgatggcaagttctttctgagacagcgcaaatgaaaccgcttctttctgggaggtgtccaggcaggtgttgaagaatgtcagcgggtgtatttcactggcaggactgggagcagatctgccaaagagcacttctatgagtgaggaggctgggccagaatgatacttctttagagcaatcagggcttttttcagtcgcctgtaagtgacatcattggcaagttttaacagtctgtaggaattctctcggtccaagctcaactggaaatcgtgggactcatcaaaggccaccgtgaccgacttctgggtgacccgggtcaagatcccagtggccagctgactgccctcattagcagcatcgtacaggcccacgatatcaccagaagtaaagctgttactgggaagagctgccgcggacccgtatcgcctgggctcaaaggtgaccagcagccgtccgtacagcccagtgcgctggctggatacctgcagcttcagcaaacacacgcctcggctctggagctctttcagagagatgttctcctgccaggacctgcgctcctccacctccgcgtctctctcgagctccagcaggtccagttgcttggtcacgaagctctccacagctgccgaggccattctagacctgcagggtttaaacgcgcgctagcggcgccgatatcttcgaaagatctggccgcctcggcccttaagaccggtggatcggccgcgggtacaattccgcagcttttagagcagaagtaacacttccgtacaggcctagaagtaaaggcaacatccactgaggagcagttctttgatttgcaccaccaccggatccgggacctgaaataaaagacaaaaaagactaaacttaccagttatctgcagaattcgcccttcgacgcgttttccggacgggttttaccacagccctctctccgagaggagggagcgcgcgcgcaaccgatgccgggaccccgcacggcagacgtcgcgccccgccctcccgaccagcctgtgtgctgctgcacctgcgcgcccgcgccccaccccttgctctttgtcgagattacccttcattggttgtggagcccaggctggggcggagccttagcggtgacgccctcaattggcaggagttcctgtctgtttaggcagggaaaagaggcggaccccattcagctgcggattggtggagttctactgtcacttggaaaaaagaggcggctagggcacagaggggctggttttgtgggcagcatttgaatgttgaggattaactgggcccttgtggactctggcgcttaaggaagtctaggctcttggcgcctattagagcctccctgctgagtagttcaccattgtgataagcatttgacttcaccagcatttctttattatcattttctgtagaagtagcaaagttgcctgttgaggagcctggcgttgttcacgcgtgaattcggtaccgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 18 - DNA, 6827 bp, Artificial Sequence
  Synthetic Polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgacggtatcgataagctttcacgtccccctctccttcctccggctggtcctctggttgctgagctcactcagcttcttatccagcctcctctgcagctgggccctcttggctgggtccagggatccgttcttggtcccgctgccggcatagaggaccccttcccggctgattctctgccgggcatgggcgcgagccctctcaccgcagccatggatctcgggcaggtggtggctgaggcagtagcggcggctgcagagctggcagaactggcccagggttgtgacgccggctgtgcacttggcaaagccgcaggtgttatcagccttaacggcggcagaaaccagggcctcaaagtcctcctccgtgggcagatctgtggccggatgtcctttggcttttttctttttcttttctggaagtttctgctgccctgaggcctgctgctccttgctggcgggctgcccctgcgcgctcctgaccctctgcagtctctccaggtgcagcgtcctcagatcaggctggtctgggccacgctgctccctgggaggctgctctgtctgcgcaggggtagggggcactggctggagaggggctgggccaccggtccctgctgggggtcccagggctgctcggggtcgcggggccctcttgctcacagtgatgaacctcctcttcccttccccagaactgtcgtgcctcagcccgtgctcctcggctatttggtggacccgcagcctgtcgtgggaattgagggaaggaggaaactccaactgcatcttcttgctggccatgaactccactatcatggcccggaagtggtccacgccatcttggctctccactccctctgggctgcctccgttgaggctgggctgagatggagcttcagaggccagagacttcccagccggcttcttccggccctgtctggcaggtgctgcagcctcctggcctccctcctgccgctggcttccggtcctggtggacgcagcaggtccctggggcttggtggcagcgtggctggaaccctgggagttctcatgggaatagttttctgggacaatatcgtcaagatactcaaaggccgtgcgtacttccccatgctgtgtgaaatactccaccagggtcttcaaaaatgcatggttgttgacagtacgggagtcacagatgaccgccacgtggcgtcgggcacgggtgacagccacgttgatcctccggtcctcagcaagaaaaccaacttcacctttcctgttggatctgacgaaggacagtatcacggcctccttctctcggccttggaagccatcgacagacttgatttcaagctcagggtgcctgtgcacaaggctctgtctgagcaggtccacctggaggttgtatggcgagaccacagcaatgtcacgggctggaacaccagcgtccaccagagcctggatgtgcaaactgacgaggcggacttcgccagggttccctttcgactgttcgtcctcctcctccagctcaaacagcccgcagccggcggtgtccaccaagagcaggggcacacccgtctcttctgtggcagccacacctgggaggtccctcaggaggtgccttgccacggaagagtgggctgtgagctgcccaaggtacatggtgtctgaggcccagcgcatgatagcctggtgcatgcggtactgcaccgtcagtgtccgcaccaccctcgcgccgtactcctcagccaggcgttccatcaggctgagtgacagtcctgccagcgcagccttgtgagagactgtggtggggggcagctgcttgtgatcgcccgccaggatgcactttctggccttcagcagggggatccagcagctcgcctcgagggcctgggcacactcgtcaatgaccaccacgtcgaagtagctctcgggcagcaacttcagggggccatcggcagacgcacctgtgtttgttgcaaggaccacgtttgccgaagtgaggctctcgagcatagctgcttcttccctctccttcagttcttttcttaacagcttaatttcatttcgaaaattacttttctctctcttatcctgggtctttttgtttttcacaaagacctggtcgatgtccttcctgatatctgcaacaatctgggcactgtcgctccgcgctaaaaccgcatccagggagtgctgctgaatggactccaggaggcgggcagggtgtcccaggcgcagaatccgctgcttacacagagccaggcgctccaccagattgtccacggcgatgttggagggggcgcagcacagaacctttaagccttgtttcacagcttgaagaatgatctcaaccacagtcgtggttttcccagtgccaggaggtccatggatgatggcaagttctttctgagacagcgcaaatgaaaccgcttctttctgggaggtgtccaggcaggtgttgaagaatgtcagcgggtgtatttcactggcaggactgggagcagatctgccaaagagcacttctatgagtgaggaggctgggccagaatgatacttctttagagcaatcagggcttttttcagtcgcctgtaagtgacatcattggcaagttttaacagtctgtaggaattctctcggtccaagctcaactggaaatcgtgggactcatcaaaggccaccgtgaccgacttctgggtgacccgggtcaagatcccagtggccagctgactgccctcattagcagcatcgtacaggcccacgatatcaccagaagtaaagctgttactgggaagagctgccgcggacccgtatcgcctgggctcaaaggtgaccagcagccgtccgtacagcccagtgcgctggctggatacctgcagcttcagcaaacacacgcctcggctctggagctctttcagagagatgttctcctgccaggacctgcgctcctccacctccgcgtctctctcgagctccagcaggtccagttgcttggtcacgaagctctccacagctgccgaggccattctagacctgcagggtttaaacgcgcgctagcggcgccgatatcttcgaaagatctggccgcctcggcccttaaaaccggtggatcggccgcgggtacaattccgcagcttttagagcagaagtaacacttccgtacaggcctagaagtaaaggcaacatccactgaggagcagttctttgatttgcaccaccaccggatccgggacctgaaataaaagacaaaaagactaaacttaccagttaactttctggtttttcagttcctcgtcgactctaggccgccaccgcggtggctgatcccgctcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggcctcgagtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgaccagaggtaccgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 19 - DNA, 130 bp, Artificial Sequence
  Synthetic Polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

(19) United States
(12) Patent Application Publication
(10) Pub. No.: US 2023/0211018 A1
(43) Pub. Date: Jul. 6, 2023

(54) MATERIALS AND METHODS FOR TREATMENT OF DISORDERS ASSOCIATED WITH THE IGHMBP2 GENE

(71) Applicants: RESEARCH INSTITUTE AT NATIONWIDE CHILDREN'S HOSPITAL, Columbus, OH (US); FONDAZIONE IRCCS CA' GRANDA OSPEDALE MAGGIORE POLICLINICO, Milan (IT); UNIVERSITÀ DEGLI STUDI DI MILANO, Milan (IT)

(72) Inventors: Kathrin Christine Meyer, Columbus, OH (US); Shibi Likhite, Columbus, OH (US); Kevin Foust, Columbus, OH (US); Brian K. Kaspar, Westerville, OH (US); Monica Nizzardo, Milan (IT); Stefania Paola Corti, Milan (IT)

(21) Appl. No.: 17/778,705

(22) PCT Filed: Nov. 23, 2020
(86) PCT No.: PCT/US2020/061863

§ 371 Date: May 20, 2022

Related U.S. Application Data
(60) Provisional application No. 62/939,270, filed on Nov. 22, 2019.

(51) Int. Cl. A61K 48/00; C12N 15/86; C12N 9/14; A61K 45/06

(52) U.S. Cl. CPC A61K 48/0066; A61K 45/06; C12N 9/14; C12N 15/86; C12Y 306/04012; C12N 2750/14143

(57) ABSTRACT

The disclosure provides gene therapy vectors, such as adeno-associated virus (AAV), designed for treatment of an immunoglobulin- µ binding protein 2 (IGHMB P2)-related disorder.

This application claims priority benefit to U.S. Provisional Application No. 62/939,270, filed Nov. 22, 2019, which is incorporated herein in its entirety.

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: 54445_Seqlisting.txt; Size: 73,110 bytes; Created: Nov. 23, 2019.

### FIELD OF THE INVENTION

The disclosure provides gene therapy vectors, such as adeno-associated virus (AAV), designed for treatment of a disorder associated with a mutation in the immunoglobulin-µ binding protein 2 (IGHMBP2) gene. The disclosed rAAV provide a wild type IGHMBP2 cDNA to a subject in need which results in expression of the wild type protein.

### BACKGROUND

The immunoglobulin-µ binding protein 2 (IGHMBP2) gene encodes a member of the Upfl-like group within the helicase superfamily 1 (SF1). This protein is known to have a helicase domain, the R3H domain, the zinc finger domain, and the nuclear localization signal sequence. IGHMBP2 is ubiquitously expressed and comprises 15 exons encoding 993 amino acids corresponding to a 110 kDa gene product. The exact role of IGHMBP2 protein in disease development is unknown. The normal IGHMBP2 is known to play a role in ribosomal RNA maturation and translation, immunoglobulin-class switching, pre-mRNA maturation, and transcription regulation by either DNA binding activity or interaction with TATA-binding protein. The IGHMPB2 protein has been classified as a member of the Upfl-like group within the helicase superfamily1 (SF1), consisting of the helicase domain, the R3H domain, the zinc finger domain, and the nuclear localization signal sequence. Autosomal recessive mutations in the IGHMPB2 gene are known to cause spinal muscular atrophy with respiratory distress type 1 (SMARD1) and Charcot-Marie-Tooth type 2S (CMT2S). The majority of the patient mutations in the IGHMPB2 gene cluster within the helicase domain and are missense mutations.

SMARD1 is an autosomal recessive motor neuron disease that is characterized by early distal lower limb muscle atrophy following proximal muscle weakness and respiratory failure. SMARD1 patients exhibit paralysis of the diaphragm between the ages of 6 weeks and 13 months. The patients usually require ventilation before 13 months of age. Loss of function mutations in the IGHMBP2 gene are known to cause SMARD1.

Charcot-Marie-Tooth (CMT) neuropathies are the most common hereditary neuropathies. CMT2 is an axonal (non-demyelinating) peripheral neuropathy characterized by distal muscle weakness and atrophy, mild sensory loss, and normal or near-normal nerve conduction velocities. CMT2 is clinically similar to CMT1, although typically less severe. Patients have slowly progressing distal muscle weakness with muscle atrophy of the upper and lower limbs. The subtypes of CMT2 are similar clinically and distinguished only by molecular genetic findings. Most subtypes of CMT2 are inherited in an autosomal dominant manner; however, some are inherited in an autosomal recessive manner. Recessive loss of function mutations in the IGHMBP2 gene are known to cause CMT2, now subclassified as CMT2S.

There are no current therapies for CMT2S and management involves treating the symptoms. Thus, there is a need to develop gene replacement therapies to treat SMARD1 and CMT2S.

### SUMMARY

In one aspect, described herein is a polynucleotide comprising (a) one or more regulatory control elements and (b) immunoglobulin-µ binding protein 2 (IGHMBP2) cDNA sequence. In some embodiments, the regulatory control element is a CBA promoter comprising a nucleotide sequence set forth in SEQ ID NO: 3, or the P546 promoter comprising a nucleotide sequence set forth in SEQ ID NO: 4 or fragments thereof which retain regulatory control or promoter activity. In some embodiments, the vector comprises the SV40 intron having the nucleotide sequence of SEQ ID NO: 5, and a fragment of the SV40 intron. In some embodiments, the IGHMBP2 cDNA comprises the polynucleotide sequence set forth in SEQ ID NO: 1.

In one embodiment, the disclosure provides for a rAAV comprising a nucleotide sequence that encodes a functional IGHMBP2 protein, wherein the nucleotide has, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 1, wherein the protein retains IGHMBP2 activity. For example, the nucleotide sequence that encodes a functional IGHMBP2 protein may comprise one or more base pair substitutions, deletions or insertions which do affect the function of the IGHMBP2. Furthermore, the nucleotide sequence that encodes a functional IGHMBP2 protein may comprise one or more base pair substitutions, deletions or insertions may increase or reduce expression of the IGHMBP2 protein, and this change in expression pattern may be desired for treatment of an IGHMBP2-related disorder, such as SMARD1 or CMT2S.

In another embodiment, the disclosure provides for a rAAV comprising a nucleotide sequence that encodes a functional IGHMBP2 protein, wherein the protein comprises an amino acid sequence that has, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 2, wherein the protein retains IGHMBP2 activity. For example, the nucleotide sequence that encodes a functional IGHMBP2 protein may comprise one or more amino acid substitutions, deletions or insertions which do affect the function of the IGHMBP2 protein.

The terms “sequence identity”, “percent sequence identity”, or “percent identical” in the context of nucleic acid or amino acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. The percentage identity of the sequences can be determined by techniques known in the art. For example, homology can be determined by a direct comparison of the sequence information between two polypeptide molecules by aligning the sequence information and using readily available computer programs such as ALIGN, ClustalW2 and BLAST. In one embodiment, when BLAST is used as the alignment tool, the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR.

In another aspect, the disclosure provides for an rAAV construct contained in the plasmid comprising the nucleotide sequence of SEQ ID NO: 7. For example, the ssAAV9.CB.IGHMBP2 vector comprises the nucleotide sequence within and inclusive of the ITR’s of SEQ ID NO: 7 and shown in FIG. 13. The rAAV vector comprises the 5′ ITR, CMV enhancer, CB promoter, a modified SV40 intron sequence, the coding sequence for the human IGHMBP2 gene, bGH polyA, and 3′ ITR. In one embodiment, the vector comprises nucleotides 1-4397 of SEQ ID NO: 7. The nucleotides within the ITRs may be in forward or reverse orientation. For example, the CMV enhancer sequence, CB promoter sequence, the SV40 sequence, human IGHMBP2 gene sequence, and bGH polyA sequence and may be in forward or reverse orientation. In another embodiment, the vector comprises a nucleotide sequence that has about at least 85%, 86%, 87%, 88%, 89%, 90%, 91%, 92%, 93%, 94% 95%, 96%, 97%, 98%, 99%, or 100% sequence identity to the nucleotides of 1-4397 of SEQ ID NO: 7. The plasmid set forth in SEQ ID NO 7 further comprises kanamycin resistance and a pUC origin of replication.

In an exemplary embodiment, the disclosure provides for an rAAV construct contained in the plasmid comprising the nucleotide sequence of SEQ ID NO: 18. For example, the ssAAV9.CB.IGHMBP2-clinical vector comprises the nucleotide sequence within and inclusive of the ITR’s of SEQ ID NO: 18 and shown in FIG. 18. The rAAV vector comprises the 5′ ITR as set out in SEQ ID NO: 19. In addition, the rAAV vector comprises the CMV enhancer, CB promoter, a modified SV40 intron sequence, the coding sequence for the human IGHMBP2 gene, bGH polyA, each in reverse orientation, and 3′ ITR as set out in SEQ ID NO: 12. In one embodiment, the vector comprises nucleotides 1-4386 of SEQ ID NO: 18. The nucleotides within the ITRs may be in forward or reverse orientation. In another embodiment, the vector comprises a nucleotide sequence that has about at least 85%, 86%, 87%, 88%, 89%, 90%, 91%, 92%, 93%, 94% 95%, 96%, 97%, 98%, 99%, or 100% sequence identity to the nucleotides of 1-4386 of SEQ ID NO: 18. The plasmid set forth in SEQ ID NO 18 further comprises kanamycin resistance gene and a pUC origin of replication. The kanamycin resistance gene may be in forward or reverse orientation.

In a further aspect, the disclosure provides for an rAAV construct contained in the plasmid comprising the nucleotide sequence of SEQ ID NO: 8. For example, the ssAAV9.P546.IGHMBP2 vector comprises the nucleotide sequence within and inclusive of the ITR’s of SEQ ID NO: 8 and shown in FIG. 14. The rAAV vector comprises the 5′ ITR, P546 promoter, a modified SV40 intron sequence, the coding sequence for the human IGHMBP2 gene, bGH polyA, and 3′ ITR. In one embodiment, the vector comprises nucleotides 1-4375 of SEQ ID NO: 8. The nucleotides within the ITRs may be in forward or reverse orientation. For example, the P546 promoter sequence, the SV40 sequence, the human IGHMBP2 gene, and bGH polyA sequence, and may be in forward or reverse orientation. In another embodiment, the vector comprises a nucleotide sequence that has about at least 85%, 86%, 87%, 88%, 89%, 90%, 91%, 92%, 93%, 94%, 95%, 96%, 97%, 98%, 99%, or 100% sequence identity to the nucleotides of 1-4397 of SEQ ID NO: 8. The plasmid set forth in SEQ ID NO 8 further comprises kanamycin resistance and a pUC origin of replication.

In an exemplary embodiment, the disclosure provides for an rAAV construct contained in the plasmid comprising the nucleotide sequence of SEQ ID NO: 17. For example, the ssAAV9.P546.IGHMBP2-clinical vector comprises the nucleotide sequence within and inclusive of the ITR’s of SEQ ID NO: 17 and shown in FIG. 16. The rAAV vector comprises the 5′ ITR as set out in SEQ ID NO: 19. In addition, the rAAV vector comprises theP546 promoter sequence, a modified SV40 intron sequence, the coding sequence for the human IGHMBP2 gene, and bGH polyA sequence, each in reverse orientation, and 3′ ITR as set out in SEQ ID NO: 12. In one embodiment, the vector comprises nucleotides 1-4364 of SEQ ID NO: 17. The nucleotides within the ITRs may be in forward or reverse orientation. In another embodiment, the vector comprises a nucleotide sequence that has about at least 85%, 86%, 87%, 88%, 89%, 90%, 91%, 92%, 93%, 94% 95%, 96%, 97%, 98%, 99%, or 100% sequence identity to the nucleotides of 1-4364 of SEQ ID NO: 17. The plasmid set forth in SEQ ID NO 17 further comprises kanamycin resistance gene and a pUC origin of replication. The kanamycin resistance gene may be in forward or reverse orientation.

In another aspect, described herein is a recombinant adeno-associated virus (rAAV) having a genome comprising a polynucleotide sequence described herein. In some embodiments, the rAAV is of the serotype AAV1, AAV2, AAV3, AAV4, AAV5, AAV6, AAV7, AAV8, AAV9, AAVRH10, AAVRH74, AAV11, AAV12, AAV13, or Anc80, AAV7m8 and their derivatives. In some embodiments, the genome of the rAAV comprises promoter fragment and an IGHMBP2 cDNA.

In some embodiments, the genome of the rAAV comprises a CBA promoter and an IGHMBP2 cDNA. An exemplary genome comprises the CBA promoter, and the IGHMBP2 cDNA such as the ssAAV9.CB.IGHMBP2, the rAAV set out as nucleotides 1-4397 of SEQ ID NO: 7 or the rAAV set out as nucleotides 1-4386 of SEQ ID NO: 18..

In some embodiments, the genome of the rAAV comprises a P546 promoter and an IGHMBP2 cDNA. An exemplary genome comprises the P546 promoter, the IGHMBP2 cDNA such as the ssAAV9.P546.IGHMBP2, the rAAV set out as nucleotides 1-4375 of SEQ ID NO: 8 or the rAAV set out as nucleotides 1-4364 of SEQ ID NO: 17..

In some embodiments, the genome of the rAAV comprises a fragment of the CBA promoter or a fragment of the P546 promoter and an IGHMBP2 cDNA, wherein the fragment of the promoter retains promoter activity.

In another aspect, described herein is an rAAV particle comprising an rAAV described herein.

Compositions comprising any of the rAAV described herein or any of the viral particles described herein. In some embodiments, the compositions further comprise an agent that increases the viscosity and/or density of the composition. For example, in some embodiments that agent is a contrast agent. The contrast agent may be 20 to 40% non-ionic, low-osmolar compound or contrast agent or about 25% to about 35% non-ionic, low-osmolar compound, such as iohexol. The disclosed composition may be formulated for any means of delivery, such as direct injection into the cerebrospinal fluid, intracerebroventricular delivery, intrathecal delivery or intravenous delivery.

In some embodiments, the composition comprises an agent that increase sthe viscosity of the composition by about 0.05%, or by about 1% or by 1.5% or about 2% or by about 2.5% or by about 3% or by about 4% or by about 5% or by about 6% or by about 7% or by about 8% or by about 9% or by about 10%. In some embodiments, an agent increases the viscosity of the composition by about 1% to about 5%, or by about 2% to 12%, or by about 5% to about 10%, or by about 1% to about 20% or by about 10% to about 20%, or by about 10% to about 30%, or by about 20% to about 40%, or by about 20% to about 50%, or by about 10% to about 50%, or by about 1% to about 50%.

In some embodiments, the composition comprises an agent that increases the density of the composition by about 0.05%, or by about 1% or by 1.5% or about 2% or by about 2.5% or by about 3% or by about 4% or by about 5% or by about 6% or by about 7% or by about 8% or by about 9% or by about 10%. In some embodiments, an agent increases the density of the composition by about 1% to about 5%, or by about 2% to 12%, or by about 5% to about 10%, or by about 1% to about 20%, or by about 10% to about 20%, or by about 10% to about 30%, or by about 20% to about 40% or by about 20% to about 50%, or by about 10% to about 50%, or by about 1% to about 50%.

For example, the disclosed composition is formulated for intrathecal delivery and comprises of a dose of rAAV or rAAV particles of about 1e13 vg per patient to about 1e15 vg per patient.

In addition, the disclosed composition is formulated for intravenous delivery and comprises of a dose of rAAV or rAAV particles of about 1e13 vg/kg to about 2e14 vg/kg.

Methods of treating an IGHMBP2-related disorder in a subject in need thereof comprising administering an rAAV or an rAAV particle described herein are specifically contemplated. In some embodiments, the methods further comprise administering an immunosuppressing agent prior to, after or simultaneously with the rAAV or rAAV particle. An IGHMPB2-related disorder includes a disorder or disease caused by a mutation that results in a loss of function of the IGHMPB2 protein or causes reduced expression of the IGHMPB2 protein. An IGHMPB2-related disorder may be any disease or disorder that is related to reduced expression or activity of the IGHMPB2 protein, despite the cause of the reduced expression or activity. In disclosure contemplates IGHMPB2-related disorders in subjects that are homozygotes for a mutation in the IGHMPB2 gene or heterozygotes for a mutation in the IGHMPB2 gene. For example, an IGHMBP2-related disorder is a neurological disorder that is associated with the presence of a mutation in the IGHMBP2 gene, such as SMARD1 or CMT2S. The IGHMBP2-related disorder also includes disorders wherein the patient has a mixed phenotype, such that the severity of the neurological disorder is between the severity observed in patients suffering from SMARD1 and CMT2S.

In any of the methods, the subject has a mutation in the IGHMBP2 gene. These mutations include those currently known, such as those set out in Table 1 or 2 herein, or a mutation(s) in the IGHMBP2 gene identified in the future that is associated with a neurological disorder.

A “subject,” as used herein, can be any animal, and may also be referred to as the patient. Preferably the subject is a vertebrate animal, and more preferably the subject is a mammal, such as a domesticated farm animal (e.g., cow, horse, pig) or pet (e.g., dog, cat). in some embodiments, the subject is a human. In some embodiments, the subject is a pediatric subject. In some embodiments, the subject is a pediatric subject, such as a subject ranging in age from 1 to 10 years. In some embodiments, the subject is 4 to 15 years of age. The subject, in on embodiment, is an adolescent subject, such as a subject ranging in age from 10 to 19 years. In other embodiments, the subject is an adult (18 years or older). In any of the disclosed methods, the rAAV or the viral particle is delivered by direct injection into the cerebrospinal fluid, intracerebroventricular delivery, intrathecal delivery or intravenous delivery. For example, in any of the methods a dose of rAAV or rAAV particles of about 1e13 vg per patient to about 1e15 vg per patient is administered by intrathecal delivery to the subject. In addition, in any of the disclosed methods, a dose of rAAV or rAAV particles of a dose of about 1e13 vg/kg to about 2e14 vg/kg is administered by intravenous delivery to the subject.

In another aspect, described herein is the use of an rAAV or an rAAV particle described herein in the preparation of a medicament for the treatment of an IGHMBP2-related disorder, such as SMARD1 or CMT2S. For example, any of the disclosed medicaments are formulated for direct injection into the cerebrospinal fluid, intracerebroventricular delivery, intrathecal delivery or intravenous delivery. For example, the medicament comprises a dose of rAAV or rAAV particles of about 1e13 vg per patient to about 1e15 vg per patient is administered by intrathecal delivery to the subject. In addition, the medicament comprises a dose of rAAV or rAAV particles of a dose of about 1e13 vg/kg to about 2e14 vg/kg is administered by intravenous delivery to the subject. In some embodiments, the medicament is administered simultaneously, prior to or after administration of an immunosuppressing agent.

In another aspect, described herein is a composition comprising an rAAV or an rAAV particle described herein for the treatment of an IGHMBP2-related disorder, such as SMARD1 or CMT2S. For example, any of the disclosed compositions are formulated for direct injection into the cerebrospinal fluid, intracerebroventricular delivery, intrathecal delivery or intravenous delivery. For example, the composition comprises a dose of rAAV or rAAV particles of about 1e13 vg per patient to about 1e15 vg per patient is administered by intrathecal delivery to the subject. In addition, the composition comprises a dose of rAAV or rAAV particles of a dose of about 1e13 vg/kg to about 2e14 vg/kg is administered by intravenous delivery to the subject. In some embodiments, the composition is administered simultaneously, prior to or after administration of an immuno-suppressing agent. In another embodiment, the composition further comprises an immuno-suppressing agent.

### BRIEF DESCRIPTION OF THE FIGURES

FIG. 1 provides schematics for AAV9.CB.IGHMBP2 (promoter 1 = CB promoter) and AAV9.P546.IGHMBP2 (promoter 2 = P546 promoter).

FIG. 2 provides a plasmid map of ssAAV.CB.IGHMBP2.Kan-Fw (SEQ ID NO: 7).

FIG. 3 provides a plasmid map of ssAAV.P546.IGHMBP2.Kan-Fw (SEQ ID NO: 8). This vector is identical to the ssAAV.P546.IGHMBP2.Kan described herein. The P5465 promoter (SEQ ID NO: 4) is also referred to as MeCp2 or 546 herein and these terms may be used interchangeably.

FIG. 4 provides representative photos of the nmd^{em3/em3} homozygous mice treated with Virus A (AAV9.CB.IGHMBP2), Virus B (empty AAV9 capsid) and Virus C (AAV9.P546.IGHMBP2). All images were taken at 2-3 weeks after treatment.

FIG. 5 provides the survival analysis of the nmd^{em3/em3} homozygous mice treated with Virus A, Virus B and Virus C up to 8 weeks post injection at which animals were sacrificed for histology. Virus A and Virus C mostly rescue the survival of these mice, while Virus B (empty viral particles) has no effect.

FIGS. 6A-6B provides graphs indicting the weight development of the nmd^{em3/em3} homozygous mice after treatment with Virus A, Virus B or Virus C.

FIG. 7 provides graphs depicting the strength testing of the nmd^{em3/em3} homozygous mice using a hanging wire test and measuring the time the mice are able to hold onto the wire until they fall after treatment with Virus A, Virus B or Virus C.

FIGS. 8A-8D provides photos demonstrating that Virus A and Virus C had a strong effect on nerve and muscle area.

FIGS. 9A-9C: FIGS. 9A and 9B provide representative photos of the fully innervated, denervated and partially innervated neuromuscular junctions (NMJ) of the medial gastrocnemius (MG) muscle 8 weeks after treatment with Virus A or Virus C. The wild type mice have fully innervated NMJ, the untreated mice had nearly no innervation in the NMJ after 2-3 weeks post birth. The treated mice had a mix of fully innervated, fragmented and de-innervated NMJ 8 weeks post-treatment. The middle panel stains for neurofilaments to represent the presynaptic nerves and the right panel stains for postsynaptic Acetylcholine receptors. FIG. 9C The graph provides counts of NMJ on medial gastrocnemius and soleus.

FIG. 10 provides a survival plot that demonstrates Virus A and Virus C improve survival in the nmd-2J mouse model.

FIGS. 11A-11D provides plots of electrophysiological outcomes: compound muscle action potential (CMAP), single motor unit potential (SMUP) and motor unit number estimation (MUNE). There is a significant increase on CMAP and MUNE with both Virus A and Virus C. Virus A and C were not different from each other CMAP: Measures strength of innervation. MUNE: Estimates number of Neurons innervating a muscle.

FIGS. 12A-12C provides results from a hanging wire test on healthy mice and on Em5 mice treated with Virus A and Virus C. Figure B shows an increase in muscle mass in healthy and Virus A treated and Virus C treated Em5 mice, and FIG. 12C provides the gastrocnemius weight in relation to total body weight.

FIG. 13 provide the annotated sequence of the plasmid ssAAV.CB.IGHMBP2.Kan-Fw (SEQ ID NO: 7).

FIG. 14 provides an annotated sequence of the plasmid ss.AAV.P546.IGHMBP2.Kan-Fw (SEQ ID NO: 8).

FIG. 15 provides a plasmid map of ssAAV.P546.IGHMBP2.Kan-Clinical (SEQ ID NO: 17). The P546 promoter (SEQ ID NO: 4) is also referred to as MeCp2 or 546 herein and these terms may be used interchangeably. In this plasmid, the P546 promoter sequence, SV40 intron sequence and the IGHMBP2 cDNA sequence are in reverse orientation.

FIG. 16 provides the annotated sequence of the plasmid ssAAV.P546.IGHMBP2.Kan-Clinical (SEQ ID NO: 17).

FIG. 17 provides a plasmid map of ssAAV.CB.IGHMBP2.Kan-Clinical (SEQ ID NO: 18). In this plasmid, the CMV enhancer sequence, the CB promoter sequence, SV40 intron sequence and the IGHMBP2 cDNA sequence are in reverse orientation.

FIG. 18 provides the annotated sequence of the plasmid ssAAV.CB.IGHMBP2.Kan-Clinical (SEQ ID NO: 18).

### DETAILED DESCRIPTION

The immunoglobulin-µ binding protein 2 (IGHMBP2) gene encodes protein that is a member of the Upfl-like group within the helicase superfamily1 (SF1), consisting of the helicase domain, the R3H domain, the zinc finger domain, and the nuclear localization signal sequence. Mutations in the IGHMPB2 gene are known to cause spinal muscular atrophy with respiratory distress type 1 (SMARD1) and Charcot-Marie-Tooth Disease type 2S (CMT2S). The majority of the patient mutations in the IGHMPB2 gene cluster within the helicase domain and are missense mutations.

### IGHMPB2 Mutations

The wild-type cDNA sequence of IGHMPB2 is set forth in SEQ ID NO: 1 (Genbank NM_002180.2) and the IGHMPB2 protein, also known as DNA-binding protein SMUB-2, is set forth in SEQ ID NO: 2 (Genbank NP_002171.2). The wild type gene product is a 993 amino acid protein that has seven putative helicase motifs and a DEAD box-like motif typical for RNA helicases. The mutations may lead to dysfunction of helicase activity. The IGHMPB2 gene is known to have 15 exons. Mutations in the IGHMPB2 gene were found to be associated with SMARD1 and CMT2S.

There are about 26 known IGHMPB2 mutation that cause SMARD1. The mutations include recessive missense mutations, nonsense mutations, frameshifts, a in-frame deletion, a frameshift insertion and a splice donor site mutation and span the 15 exons of the IGHMPB2 gene (Luan et al., Brain & Dev. 28: 685-689, 2016; incorporated herein by reference). Exemplary mutations known to cause SMARD1 are summarized below in Table 1. The disclosed gene therapy vectors and methods of treatment are not limited to disorders caused by the mutations provided in Table 1 or those that are known at the time of filing as other mutations of the IGHMPB2 may be identified in the future that cause SMARD1.

Table 1

| | |
|---|---|
| cDNA Nucleotide Change | Protein Amino Acid Change |
| c.344C>T | p.115T>M |
| | p.C496X |
| | p.D565N |
| c.1737C>A | p.579F>L |
| | p.R603C |
| | p.R605X |
| c.1082T>C | |
| c.1478C>T | p.Thr493I1e |
| c.676G>T | |
| c.2083A>T | |
| IVS 13 + 1G>T | |
| c.1144G>A | |
| c.2598 2601del | |
| c.138T>A | |
| c.2911 2912del | |
| c.604T>G | |
| c.1591C>A | |
| c.1738G>A | |
| c.1813C>T | |
| c.2770C>T | |
| c.238A>G | |
| c.1488C>A | p.C496X |
| c.1156T>C | |
| c.2968_2980del(Hom) | |
| c.1118T>G | |
| c.1582G>A | |
| c.734A>G (Het) | |
| c.1813C>T (Het) +deletion | |
| c.1730T>C | p.L577P |

The known IGHMPB2 mutation causing CMT2S are an autosomal recessive mutation that causes axonal neuropathy (Cottenie et al., Am J Hum Genet. 2014;95:590-601; Schottmann et al., Neurology. 2015;84:523-31, both incorporated herein by reference). Exemplary mutations known to cause CMT2S are summarized in Table 2 below. The disclosed gene therapy vectors and methods of treatment are not limited to disorders caused by the mutations provided in Table 2 or those that are known at the time of filing as other mutations of the IGHMPB2 may be identified in the future that cause CMT2S.

TABLE 2

| | |
|---|---|
| cDNA Nucleotide Change | Protein Amino Acid Change |
| c.138T>A | p.Cys46Ter |
| c.604T>G | p.Phe202Val |
| c.2911 2912delAG | p.Arg97GLusTer4 |
| c.1591C>A | p.Pro531Thr |
| c.1738G>A | p.Val580Ile |
| c.449+1G>T | |
| c.2784+lG>T | |

### Diagnosis and Progression of SMARD1

SMARD1 is also known as autosomal recessive distal spinal muscular atrophy 1 distal hereditary motor neuronopathy type VI (dHMN6 or HMN6) or distal muscular dystrophy type 1 (DSMA-1). This disorder is a variant of infantile SMA. The most prominent symptoms of SMARD 1 are severe respiratory distress resulting from diaphragmatic paralysis with eventration shown on chest x-ray, low birth weight below the 3^{rd} centile, inability to wean and progressive muscle weakness in the upper limbs and distal muscles are also affected. Additional symptoms include low motor nerve conduction velocities, and a reduction in the size of myelinated fibers on sural nerve biopsy, Sensory and autonomic nerves were also affected in some patients, as demonstrated by decreased pain perception, excessive sweating, constipation, and bladder incontinence. Clinical features include: intrauterine growth retardation, prematurity, weak cry, and foot deformities. The symptoms usually present at age 1 month to 6 months.

### Diagnosis and Progression of Charcot-Marie-Tooth Hereditary Neuropathy 2 (CMT2)

CMT2 is a progressive peripheral motor and sensory neuropathy and it is generally diagnoses as measuring one or more of i) nerve conduction velocities (NCVs) that are with the normal range (>40-50 m/s) although occasionally in mildly abnormal range (30-40 m/s), ii) EMG testing that shows evidence of axonal neuropathy with such findings as positive waves, polyphasic potentials, or fibrillations and reduced amplitudes of evoked motor and sensory responses, iii) greatly reduced compound motor action potentials (CMAP) and/or family history that is typically (but not always) consistent with recessive manner.

A nerve biopsy is not required for diagnosis but it may be used as a method of monitoring progression or confirming diagnosis. Nerve biopsies show loss of myelinated fibers with signs of regeneration, axonal sprouting, and atrophic axons with neurofilaments, and large nodal gaps and shorter internodal lengths than controls, suggesting a developmental abnormality of internode formation.

CMT2S more prominently involves the nerves of the motor system rather than the sensory system, although both are involved. The affected individual typically has slowly progressive weakness and atrophy of distal muscles in the feet and/or hands usually associated with depressed tendon reflexes and mild or no sensory loss. Affected individuals usually become symptomatic between ages five and 25 years, though onset ranges from infancy with delayed walking to after the third decade. The typical presenting symptom is weakness of the feet and ankles. The initial physical findings are depressed or absent tendon reflexes with weakness of foot dorsiflexion at the ankle.

The adult patients with CMT2S typically have bilateral foot drop, symmetric atrophy of muscles below the knee (stork leg appearance) and absent tendon reflexes in the lower extremities. Brisk tendon reflexes and extensor plantar responses have also been reported as well as asymmetric muscle atrophy in up to 15% of affected individuals. Vocal cord or phrenic nerve involvement resulting in difficulty with phonation or breathing has been observed. In addition, restless leg syndrome and sleep apnea have also been observed.

### AAV Gene Therapy

The present disclosure provides for gene therapy vectors, e.g. rAAV vectors, expressing the IGHMPB2 cDNA and methods of treating an IGHMPB2-related disorder. The IGHMPB2-related disorder includes disorders caused by a mutation that causes a loss of function of the IGHMPB2 protein or causes reduced expression of the IGHMPB2 protein. Furthermore, any disease or disorder that is related to reduced expression or activity of the IGHMPB2 protein, despite the cause of the reduced expression or activity.

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle.

Adeno-associated virus (AAV) is a replication-deficient parvovirus, the single-stranded DNA genome of which is about 4.7 kb in length including an inverted terminal repeat (ITRs). Exemplary ITR sequences may be 130 base pairs in length or 141 base pairs in length, such as the ITR sequence set out in SEQ ID NOS: 11, 12 and 19. There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cloning of the AAVrh.74 serotype is described in Rodino-Klapac., et al. Journal of translational medicine 5, 45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° C. to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics.

Recombinant AAV genomes of the disclosure comprise nucleic acid molecule of the disclosure and one or more AAV ITRs flanking a nucleic acid molecule. AAV DNA in the rAAV genomes may be from any AAV serotype for which a recombinant virus can be derived including, but not limited to, AAV serotypes (e.g., AAV1, AAV2, AAV3, AAV4, AAV5, AAV6, AAV7, AAV8, AAV9, AAVRH10, AAVRH74, AAV11, AAV12, AAV13, or Anc80, AAV7m8 and their derivatives). Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art.

The provided recombinant AAV (i.e., infectious encapsidated rAAV particles) comprise a rAAV genome. The term “rAAV genome” refers to a polynucleotide sequence that is derived from a native AAV genome that has been modified. In some embodiments, the rAAV genome has been modified to remove the native cap and rep genes. In some embodiments, the rAAV genome comprises the endogenous 5′ and 3′ inverted terminal repeats (ITRs). In some embodiments, the rAAV genome comprises ITRs from an AAV serotype that is different from the AAV serotype from which the AAV genome was derived. In some embodiments, the rAAV genome comprises a transgene of interest flanked on the 5′ and 3′ ends by inverted terminal repeat (ITR). In some embodiments, the rAAV genome comprises a “gene cassette.” In exemplary embodiments, the genomes of both rAAV lack AAV rep and cap DNA, that is, there is no AAV rep or cap DNA between the ITRs of the genomes.

The rAAV genomes provided herein, in some embodiments, comprise one or more AAV ITRs flanking the transgene polynucleotide sequence. The transgene polynucleotide sequence is operatively linked to transcriptional control elements (including, but not limited to, promoters, enhancers and/or polyadenylation signal sequences) that are functional in target cells to form a gene cassette. Examples of promoters are the pIRF promoter, chicken β actin promoter (CBA) comprising the polynucleotide sequence set forth in SEQ ID NO: 3, and the P546 promoter comprising the polynucleotide sequence set forth in SEQ ID NO: 4. Additional promoters are contemplated herein including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter.

Additionally provided herein are a CB promoter sequence, a P546 promoter sequence, and promoter sequences at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of the CBA (SEQ ID NO: 3) or P546 (SEQ ID NO: 4) sequence which exhibit transcription promoting activity.

Other examples of transcription control elements are tissue specific control elements, for example, promoters that allow expression specifically within neurons or specifically within astrocytes. Examples include neuron specific enolase and glial fibrillary acidic protein promoters. Inducible promoters are also contemplated. Non-limiting examples of inducible promoters include, but are not limited to a metallothionine promoter, a glucocorticoid promoter, a progesterone promoter, and a tetracycline-regulated promoter. The gene cassette may also include intron sequences to facilitate processing of a transgene RNA transcript when expressed in mammalian cells. One example of such an intron is the SV40 intron.

rAAV genomes provided herein comprises a polynucleotide (SEQ ID NO: 1) encoding IGHMPB2 protein. In some embodiments, the rAAV genomes provided herein comprises a polynucleotide that encodes a polypeptide comprising an amino acid sequence that is at least: 90%, 91%, 92%, 93%, 94%, 95%, 96%, 97%, 98%, or 99% identical to the amino acid sequence encoded by the IGHMPB2 cDNA (SEQ ID NO 1).

rAAV genomes provided herein comprises a nucleotides 1- 4397 of SEQ ID NO: 7 or nucleotides 1-4375 of SEQ ID NO: 8. In some embodiments, the rAAV genomes provided herein comprises a polynucleotide that at least: 90%, 91%, 92%, 93%, 94%, 95%, 96%, 97%, 98%, or 99% identical to the nucleotide sequences of nucleotides 1- 4397 of SEQ ID NO: 7 or nucleotides 1- 4375 of SEQ ID NO: 7 SEQ ID NO: 7 or 8.

rAAV genomes provided herein, in some embodiments, a polynucleotide sequence that encodes an IGHMPB2 protein and that hybridizes under stringent conditions to the polynucleotide sequence set forth in SEQ ID NO: 1 or the complement thereof.

DNA plasmids of the disclosure comprise rAAV genomes of the disclosure. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-9, AAV-1, AAV-2, AAV-3, AAV-4, AAV-5, AAV-6, AAV-7, AAVrh.74, AAV-8, AAV-10, AAV-11, AAV-12 and AAV-13. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., Mol. Cell. Biol., 7:349 (1988). Samulski et al., J. Virol., 63:3822-3828 (1989); U.S. Patent No. 5,173,414; WO 95/13365 and corresponding U.S. Pat. No. 5,658.776 ; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13:1244-1250 (1995); Paul et al. Human Gene Therapy 4:609-615 (1993); Clark et al. Gene Therapy 3:1124-1132 (1996); U.S. Pat.. No. 5,786,211; U.S. Pat. No. 5,871,982; and U.S. Pat. No. 6,258,595. The foregoing documents are hereby incorporated by reference in their entirety herein, with particular emphasis on those sections of the documents relating to rAAV production.

The disclosure thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69427-443 (2002); U.S. Patent No. 6,566,118 and WO 98/09657.

Compositions provided herein comprise rAAV and a pharmaceutically acceptable excipient or excipients. Acceptable excipients are nontoxic to recipients and are preferably inert at the dosages and concentrations employed, and include, but are not limited to, buffers such as phosphate [e.g., phosphate-buffered saline (PBS)], citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, copolymers such as poloxamer 188, pluronics (e.g., Pluronic F68) or polyethylene glycol (PEG). Compositions provided herein can comprise a pharmaceutically acceptable aqueous excipient containing a non-ionic, low-osmolar compound such as iobitridol, iohexol, iomeprol, iopamidol, iopentol, iopromide, ioversol, or ioxilan, where the aqueous excipient containing the non-ionic, low-osmolar compound can have one or more of the following characteristics: about 180 mgI/mL, an osmolality by vapor-pressure osmometry of about 322 mOsm/kg water, an osmolarity of about 273 mOsm/L, an absolute viscosity of about 2.3 cp at 20° C. and about 1.5 cp at 37° C., and a specific gravity of about 1.164 at 37° C.

Exemplary compositions comprise an agent to increase the viscosity and/or density of the composition. For example, the composition comprises a contrast agent to increase the viscosity and/or density of the composition. Exemplary compositions comprise about 20 to 40% non-ionic, low-osmolar compound or contrast agent or about 25% to about 35% non-ionic, low-osmolar compound. An exemplary composition comprises scAAV or rAAV viral particles formulated in 20 mM Tris (pH8.0), 1 mM MgCl₂, 200 mM NaCl, 0.001% poloxamer 188 and about 25% to about 35% non-ionic, low-osmolar compound. Another exemplary composition comprises scAAV formulated in and 1X PBS and 0.001% Pluronic F68.

Dosages of rAAV to be administered in methods of the disclosure will vary depending, for example, on the particular rAAV, the mode of administration, the time of administration, the treatment goal, the individual, and the cell type(s) being targeted, and may be determined by methods standard in the art. Dosages may be expressed in units of viral genomes (vg). Dosages contemplated herein include about 1×10⁷, 1×10⁸, 1×10⁹ ,5×10⁹, 6 ×10⁹ , 7×10⁹, 8×10⁹, 9×10⁹, 1×10¹⁰, 2×10¹⁰, 3×10¹⁰, 4×10¹⁰, 5×10¹⁰, 1×10¹¹, about 1×10¹², about 1×10¹³, about 1.1×10¹³, about 1.2×10¹³, about 1.3×10¹³, about 1.5×10¹³, about 2×10¹³, about 2.5×10¹³, about 3×10¹³, about 3.5×10¹³, about 4×10¹³, about 4.5×10¹³, about 5× 10¹³, about 6×10¹³, about 1×10¹⁴, about 2×10¹⁴, about 3×10¹⁴, about 4×10¹⁴about 5×10¹⁴, about 1×10¹⁵, to about 1×10¹⁶, or more total viral genomes.

Dosages of about 1×10⁹ to about 1×10¹⁰, about 5×10⁹ to about 5×10¹⁰, about 1×10¹⁰ to about 1×10¹¹, about 1×10¹¹ to about 1×10¹⁵ vg, about 1×10¹² to about 1×10¹⁵ vg, about 1×10¹² to about 1×10¹⁴ vg, about 1×10¹³ to about 6×10¹⁴ vg, about 1×10¹³ to about 1×10¹⁵ vg and about 6×10¹³ to about 1.0×10¹⁴ vg are also contemplated. One dose exemplified herein is 1×10¹³ vg administered via intrathecal delivery. Another dose exemplified herein is 1.5×10¹³ vg administered via intrathecal delivery.

Dosages are also may be expressed in units of vg/kg. Dosages contemplated herein include about 1×10⁷ vg/kg, 1×10⁸ vg/kg, 1×10⁹ vg/kg, 5×10⁹ vg/kg, 6×10⁹ vg/kg, 7×10⁹ vg/kg, 8×10⁹ vg/kg, 9×10⁹ vg/kg, 1×10¹⁰ vg/kg, 2×10¹⁰ vg/kg¹⁰, 3×10¹⁰ vg/kg, 4×10¹⁰ vg/kg, 5×10¹⁰ vg/kg, 1×10¹¹ vg/kg, about 1×10¹² vg/kg, about 1×10¹³ vg/kg, about 1.1×10¹³ vg/kg, about 1.2x10¹³ vg/kg, about 1.3x10¹³ vg/kg, about 1.5x10¹³ vg/kg, about 2x10¹³ vg/kg, about 2.5 x10¹³ vg/kg, about 3 x10¹³ vg/kg, about 3.5 x10¹³ vg/kg, about 4x10¹³ vg/kg, about 4.5x 10¹³ vg/kg, about 5 x10¹³ vg/kg, about 6x10¹³ vg/kg, about 1x10¹⁴ vg/kg, about 2 x10¹⁴ vg/kg, about 3 x10¹⁴ vg/kg, about 4x10¹⁴ vg/kg about 5x10¹⁴ vg/kg, about 1x10¹⁵ vg/kg, to about 1x10¹⁶ vg/kg.

Dosages of about 1x10⁹ vg/kg to about 1 x10¹⁰ vg/kg, about 5x10⁹ vg/kg to about 5 x10¹⁰ vg/kg, about 1x10¹⁰ vg/kg to about 1x10¹¹ vg/kg, about 1x10¹¹ vg/kg to about 1x10¹⁵ vg/kg, about 1x10¹² vg/kg to about 1x10¹⁵ vg/kg, about 1x10¹² vg/kg to about 1x10¹⁴ vg/kg, about 1x10¹³ vg/kg to about 2x10¹⁴ vg/kg, about 1x10¹³ vg/kg to about 1x10¹⁵ vg/kg and about 6x10¹³ vg/kg to about 1.0x10¹⁴ vg/kg are also contemplated. One dose exemplified herein is 1x10¹³ vg/g administered via intravenous delivery. Another dose exemplified herein is 2.5x10¹⁴ vg/kg administered via intravenous delivery.

Methods of transducing a target cell with rAAV, in vivo or in vitro, are contemplated by the disclosure. The in vivo methods comprise the step of administering an effective dose, or effective multiple doses, of a composition comprising a rAAV of the disclosure to an animal (including a human being) in need thereof. If the dose is administered prior to development of a disorder/disease, the administration is prophylactic. If the dose is administered after the development of a disorder/disease, the administration is therapeutic. In embodiments of the disclosure, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. Example of a disease contemplated for prevention or treatment with methods of the disclosure is SMARD1 and CMT2S.

Combination therapies are also contemplated by the disclosure. Combination as used herein includes both simultaneous treatment and sequential treatments. Combinations of methods of the disclosure with standard medical treatments are specifically contemplated, as are combinations with novel therapies. In some embodiments, the combination therapy comprises administering an immunosuppressing agent in combination with the gene therapy disclosed herein.

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the disclosure may be chosen and/or matched by those skilled in the art taking into account the infection and/or disease state being treated and the target cells/tissue(s) that are to express the wild type IGHMPB2 protein.

The disclosure provides for local administration and systemic administration of an effective dose of rAAV and compositions of the disclosure. For example, systemic administration is administration into the circulatory system so that the entire body is affected. Systemic administration includes enteral administration such as absorption through the gastrointestinal tract and parenteral administration through injection, infusion or implantation.

Transduction of cells with rAAV of the disclosure results in sustained expression of the IGHMPB2 protein. The present disclosure thus provides methods of administering/delivering rAAV which express IGHMPB2 protein to an animal, preferably a human being. These methods include transducing cells with one or more rAAV of the present disclosure.

The term “transduction” is used to refer to the administration/delivery of the coding region of the IGHMPB2 to a recipient cell either in vivo or in vitro, via a replication-deficient rAAV of the disclosure resulting in expression of IGHMPB2 the recipient cell.

### Immunosuppressing Agents

The immunosuppressing agent may be administered before or after the onset of an immune response to the rAAV in the subject after administration of the gene therapy. In addition, the immunosuppressing agent may be administered simultaneously with the gene therapy or the protein replacement therapy. The immune response in a subject includes an adverse immune response or an inflammatory response following or caused by the administration of rAAV to the subject. The immune response may be the production of antibodies in the subject in response to the administered rAAV.

Exemplary immunosuppressing agents include glucocorticosteroids, janus kinase inhibitors, calcineurin inhibitors, mTOR inhibitors, cyctostatic agents such as purine analogs, methotrexate and cyclophosphamide, inosine monophosphate dehydrogenase (IMDH) inhibitors, biologics such as monoclonal antibodies or fusion proteins and polypeptides, and di peptide boronic acid molecules, such as Bortezomib.

The immunosuppressing agent may be an anti-inflammatory steroid, which is a steroid that decreases inflammation and suppresses or modulates the immune system of the subject. Exemplary anti-inflammatory steroid are glucocorticoids such as prednisolone, betamethasone, dexamethasone, hydrocortisone, methylprednisolone, deflazacort, budesonide or prednisone.

Janus kinase inhibitors are inhibitors of the JAK/STAT signaling pathway by targeting one or more of the Janus kinase family of enzymes. Exemplary janus kinase inhibitors include tofacitinib, baricitinib, upadacitinib, peficitinib, and oclacitinib.

Calcineurin inhibitors bind to cyclophilin and inhibits the activity of calcineurin Exemplary calcineurine inhibitors includes cyclosporine, tacrolimus and picecrolimus.

mTOR inhibitors reduce or inhibit the serine/threonine-specific protein kinase mTOR. Exemplary mTOR inhibitors include sirolimus, everolimus, and temsirolimus.

The immunosuppressing agents include immune suppressing macrolides. The term “immune suppressing macrolides” refer to macrolide agents that suppresses or modulates the immune system of the subject. A macrolide is a class of agents that comprise a large macrocyclic lactone ring to which one or more deoxy sugars, such as cladinose or desoamine, are attached. The lactone rings are usually 14-, 15-, or 16-membered. Macrolides belong to the polyketide class of agents and may be natural products. Examples of immunosuppressing macrolides include tacrolimus, pimecrolimus, and sirolimus.

Purine analogs block nucleotide synthesis and include IMDH inhibitors. Exemplary purine analogs include azathioprine, mycophenolate and lefunomide.

Exemplary immunosuppressing biologics include abatacept, adalimumab, anakinra, certolizumab, etanercept, golimumab, infliximab, ixekizumab, natalizumab, rituximab, secukinumab, tocilizumab, ustekinenumab, vedolizumab, basiliximab, belatacep, and daclizumab.

In particular, the immunosuppressing agent is an anti-CD20 antibody. The term anti-CD20 specific antibody refers to an antibody that specifically binds to or inhibits or reduces the expression or activity of CD20. Exemplary anti-CD20 antibodies include rituximab, ocrelizumab or ofatumumab.

Additional examples of immuosuppressing antibodies include anti-CD25 antibodies (or anti-IL2 antibodies or anti-TAC antibodies) such as basiliximab and daclizumab, and anti-CD3 antibodies such as muromonab-CD3, otelixizumab, teplizumab and visilizumab, anti-CD52 antibodies such as alemtuzumab.

The following EXAMPLES are provided by way of illustration and not limitation. Described numerical ranges are inclusive of each integer value within each range and inclusive of the lowest and highest stated integer.

### EXAMPLES

### Example 1 - Gene Therapy Constructs Encoding IGHMBP2

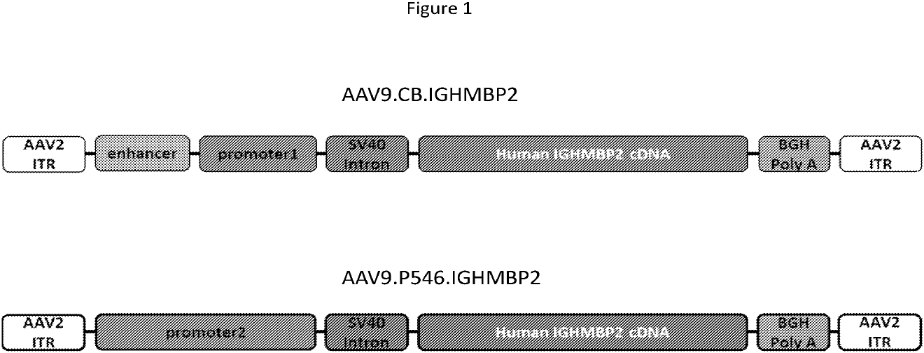

AAV genome constructs encoding IGHMBP2 were generated as set forth in FIG. 1, which depicts the AAV9 vector design with the full-length transcript of IGHMBP2 cDNA under the control of ubiquitous promoters. The promoters contemplated for inclusion in these constructs are either i) the cmv-enhancer chicken beta actin promoter (CBA; SEQ ID NO: 3) or a synthetic truncated methyl CpG binding protein 2 (MeCp2) promoter referred to as P546 (SEQ ID NO: 4) or 546.

A human GFP cDNA clone was obtained from Origene, Rockville, MD. The IGHMBP2 cDNA alone was further subcloned into a self-complementary AAV9 genome under the control of one or more of either i) the P546 promoter or v) the hybrid chicken β-Actin promoter (CB). The plasmid construct also included an intron such as the simian virus 40 (SV40) chimeric intron, and a Bovine Growth Hormone (BGH) polyadenylation signal (BGH PolyA). The constructs were packaged into either AAV9 genome.

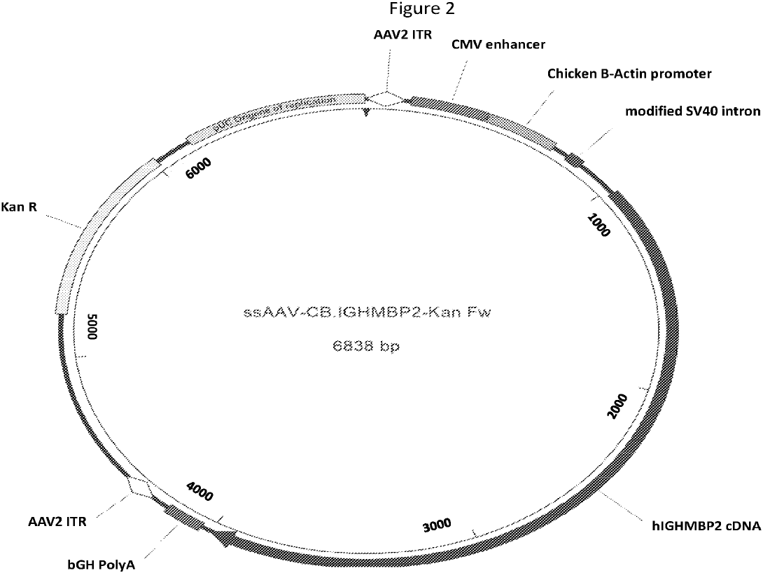

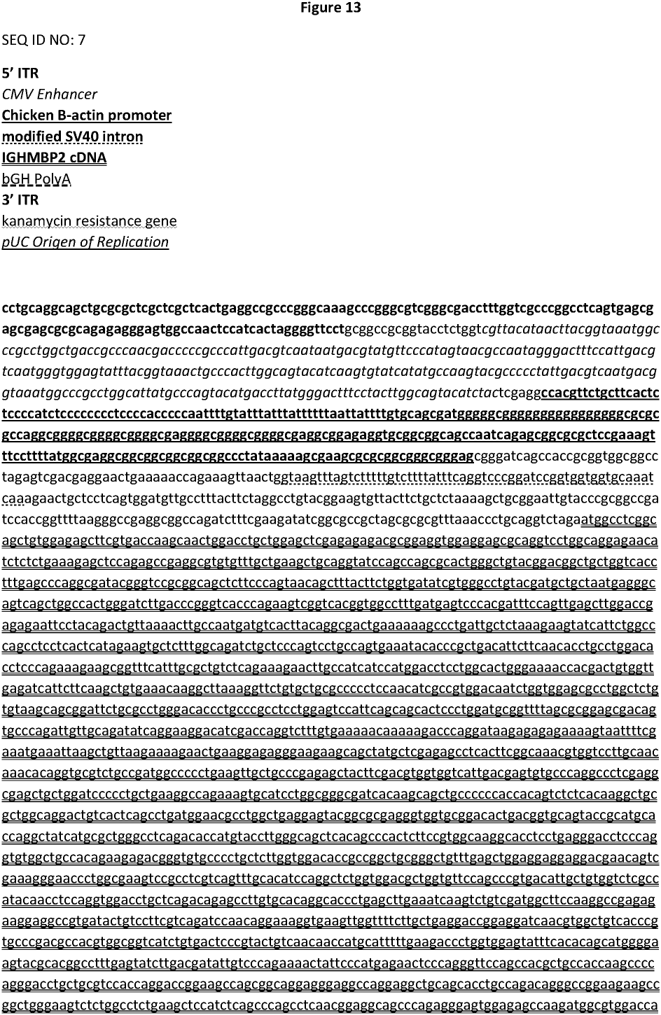

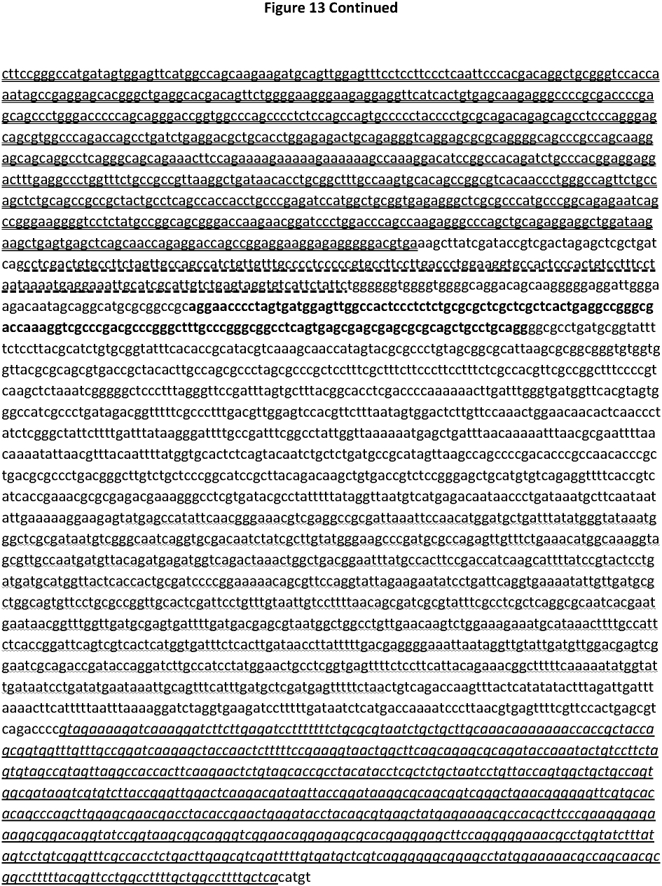

The map for plasmid ssAAV.CB.IGHMBP2.Kan.-Fw (the kanamycin resistance gene is in the forward orientation) is set out in FIG. 2 and the sequence of the entire plasmid is provided in SEQ ID NO: 7. The ssAAV.CB.IGHMBP2 vector comprises the nucleotide sequence within and inclusive of the ITR’s of SEQ ID NO: 7 and as shown in FIG. 13. The rAAV vector comprises the 5′ AAV2 ITR, CMV enhancer, CBA promoter, a modified SV40 intron sequence, the coding sequence for the IGHMBP2 gene, bGH polyA, and 3′ AAV2 ITR. The plasmid set forth in SEQ ID NO: 7 further comprises kanamycin resistance with pUC origin of replication. Plasmid ssAAV.CB.IGHMBP2.Kan.-Rv (in which the Kanamycin resistance gene is in reverse orientation) is provided as SEQ ID NO: 9.

Table 2 shows the molecular features of the plasmid ssAAV.CB.IGHMBP2.Kan.-Fw (SEQ ID NO: 7), in which range refers to the nucleotides in SEQ ID NO: 7 and ► indicates the kanamycin gene is in the forward orientation.

TABLE 2

| Name | Range ▲ | Strand | Length |
|---|---|---|---|
| AAV2 ITR | 1..141 | ► | 141 |
| CMV enhancer | 162..441 | ► | 280 |
| Chicken B-Actin promoter | 448..717 | ► | 270 |
| Modified SV40 intron | 783..842 | ► | 60 |
| hlGHMBP2 cDNA | 1021..4002 | ► | 2982 |
| bGH PolyA | 4041..4187 | ► | 147 |
| AAV2 ITR | 4257..4397 | ► | 141 |
| Kan R | 5209..6018 | ► | 810 |
| pUC Origene of replication | 6166..6833 | ► | 668 |

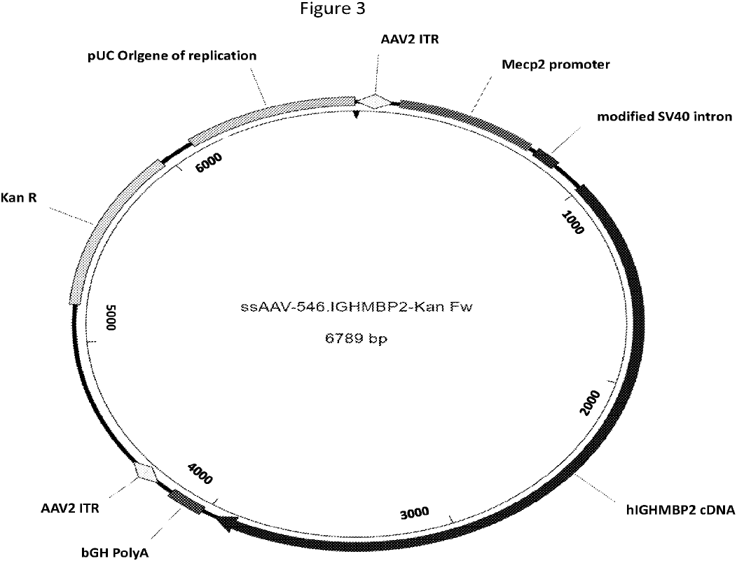

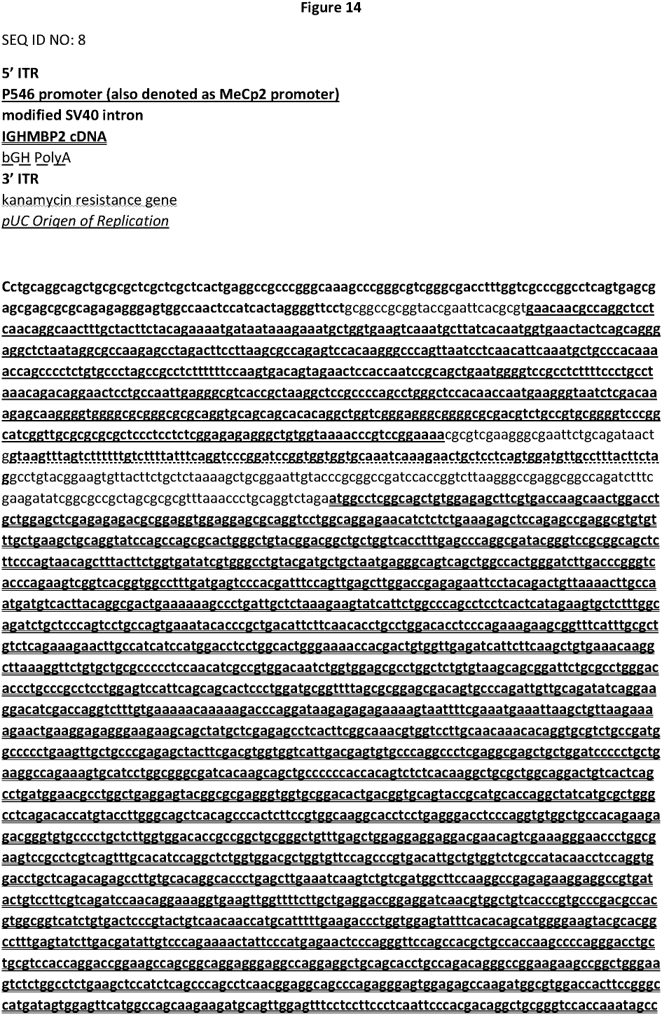

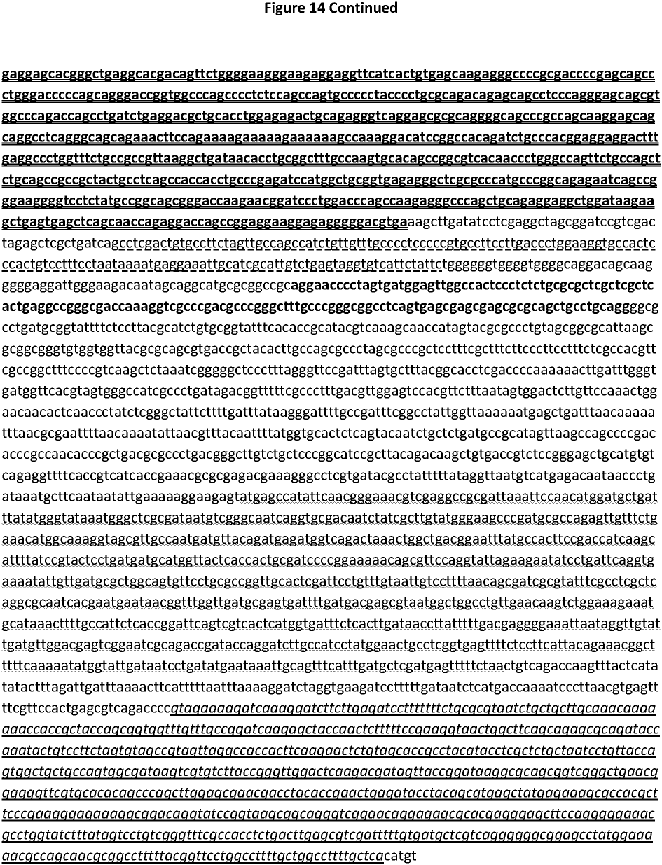

The map for plasmid ssAAV.P546.IGHMBP2.Kan.-Fw (kanamycin resistance gene is in the forward orientation) is set out in FIG. 3 and the sequence of the entire plasmid is provided in SEQ ID NO: 8. The ssAAV.P546.IGHMBP2 vector comprises the nucleotide sequence within and inclusive of the ITR’s of SEQ ID NO: 8 and as shown in FIG. 14. The rAAV vector comprises the 5′ AAV2 ITR, P546 promoter (also denoted herein as MeCp2 promoter or P546 promoter), a modified SV40 intron sequence, the coding sequence for the IGHMBP2 gene, bGH polyA, and 3′ AAV2 ITR. The plasmid set forth in SEQ ID NO:8 further comprises kanamycin resistance with pUC origin of replication. Plasmid ssAAV.P546.IGHMBP2.Kan.-Rv (in which the kanamycin gene is in reverse orientation) is provided as SEQ ID NO: 10.

Table 3 shows the molecular features of the plasmid ssAAV.P546.IGHMBP2.Kan.-Fw (SEQ ID NO: 8), in which range refers to the nucleotides in SEQ ID NO: 8 and the ► indicates the kanamycin resistance gene in the forward orientation.

TABLE 3

| Name | Range ▲ | Strand | Length |
|---|---|---|---|
| AAV2 ITR | 1..141 | ► | 141 |
| Mecp2 promoter | 168..713 | ► | 546 |
| Modified SV40 intron | 745..842 | ► | 98 |
| hlGHMBP2 cDNA | 984..3965 | ► | 2982 |
| bGH PolyA | 4019..4165 | ► | 147 |
| AAV2 ITR | 4235..4375 | ► | 141 |
| Kan R | 5187..5996 | ► | 810 |
| pUC Origene of replication | 6144..6811 | ► | 668 |

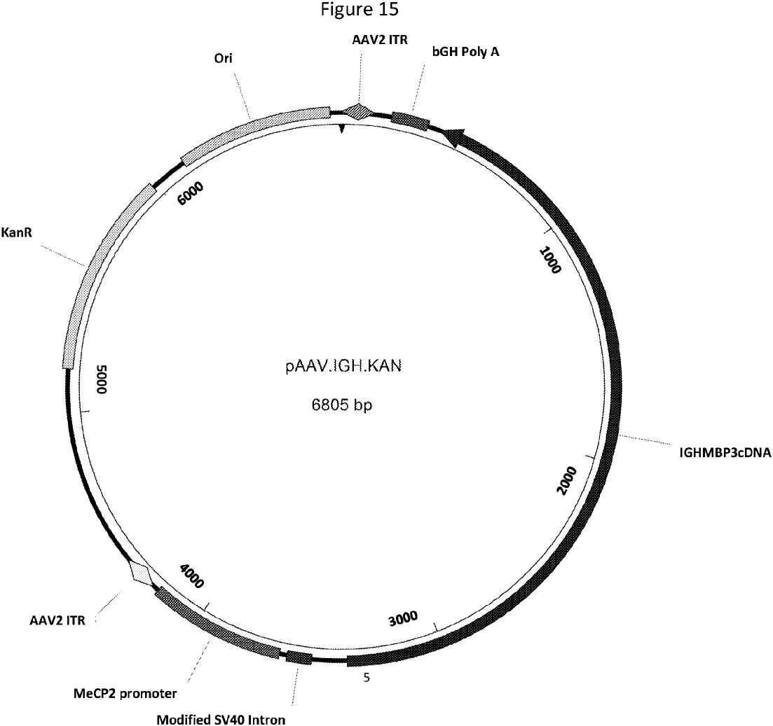

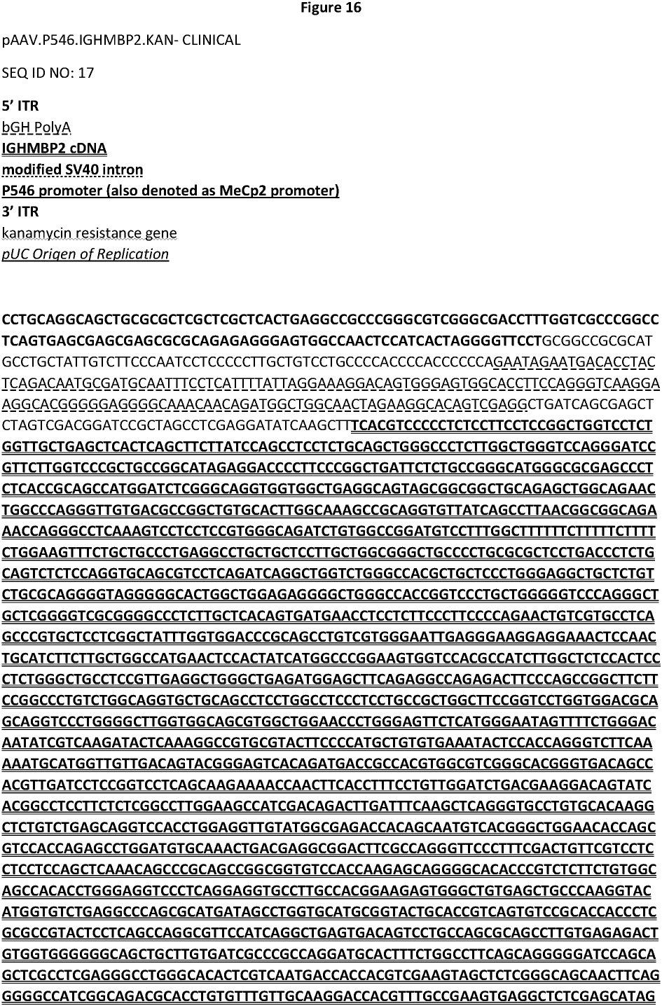

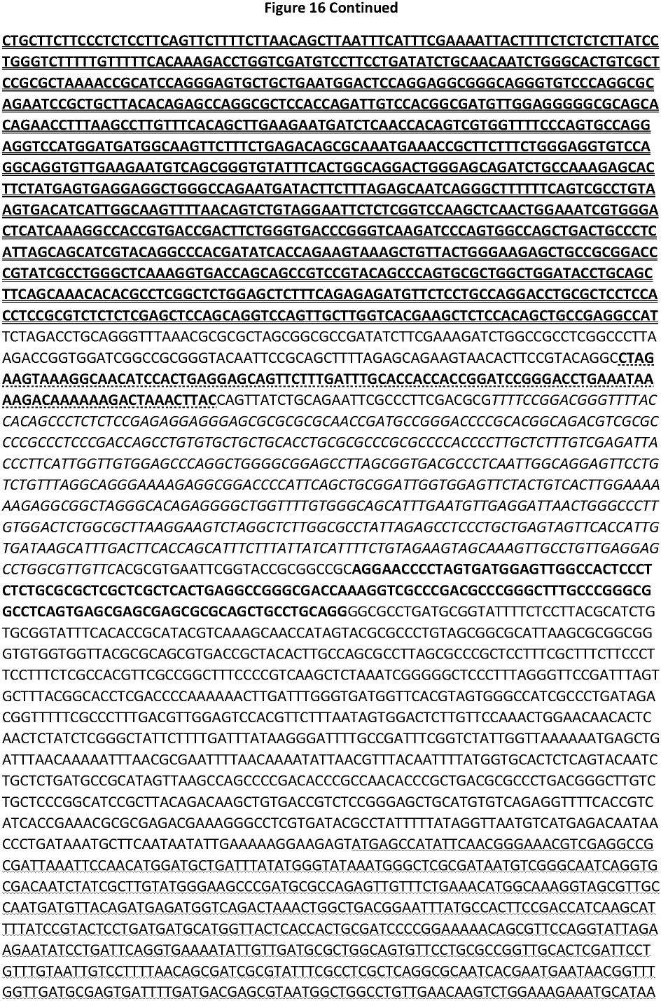

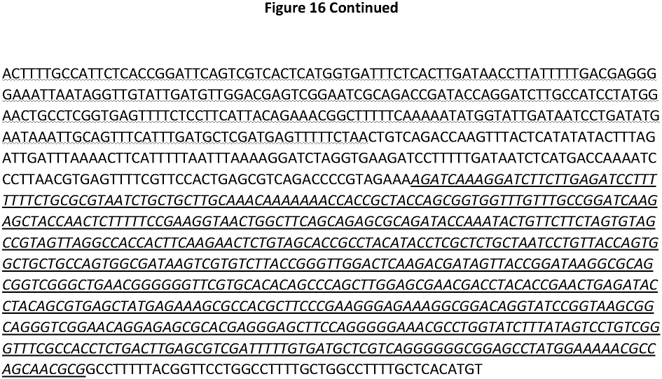

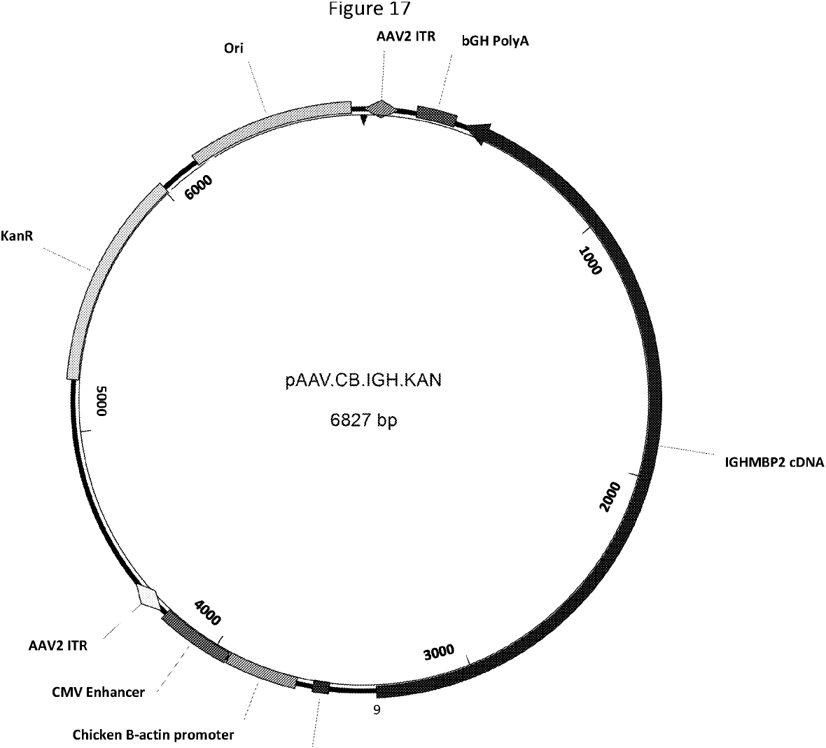

The map for plasmid ssAAV.P546.IGHMBP2.Kan-clinical (the P546 promoter sequence, IGHMBP2 cDNA sequence, the SV40 intron and the bGH polyadenylation sequence are in reverse orientation) is set out in FIG. 15 and the sequence of the entire plasmid is provided in SEQ ID NO: 17. The ssAAV.P546.IGHMBP2-clinical vector comprises the nucleotide sequence within and inclusive of the ITR’s, and as shown in FIG. 16. The rAAV vector comprises the 5′ AAV2 ITR (SEQ ID NO: 19), P546 promoter, a modified SV40 intron sequence, the coding sequence for the IGHMBP2 gene, bGH polyA, and 3′ AAV2 ITR (SEQ ID NO: 12). The plasmid set forth in SEQ ID NO: 17 further comprises kanamycin resistance with pUC origin of replication. The kanamycin resistance gene is in the forward orientation in FIG. 15, but plasmids with the kanamycin resistance gene in the reverse orientation are also contemplated.

Table 4 shows the molecular features of the plasmid ssAAV.P546.IGHMBP2.Kan- clinical (SEQ ID NO: 17), in which range refers to the nucleotides in SEQ ID NO: 17 and ► indicates the element is in the forward orientation and the ◄ indicates the element is in the reverse orientation.

TABLE 4

| Type | Name | Range | Strand | Length | Description |
|---|---|---|---|---|---|
| repeat_region | AAV2 ITR | 4224..4364 | ► | 141 | inverted terminal repeat of adeno-associated virus serotype 2 |
| misc_feature | AAV2 ITR | 1..130 | ► | 130 | AAV2 ITR |
| misc_feature | IGHMBP2 cDNA | Complement (400..3381) | ◄ | 2982 | IGHMBP2 cDNA |
| CDS | KanR | 5176..5985 | ► | 810 | aminoglycoside phosphotransferase |
| misc_feature | MeCP2 promoter | Complement (3652..4197) | ◄ | 546 | MeCP2 promoter |
| intron | Modifed SV40 intron | Complement (3523..3620) | ◄ | 98 | modified SV40 intron with splice donor and acceptor sites |
| rep_origin | Ori | 6140..6759 | ► | 620 | high-copy-number ColEl/pMBl/pBR322/pUC origin of replication |
| misc_feature | bGH PolyA | Complement (200..346) | ◄ | 147 | bGH PolyA |

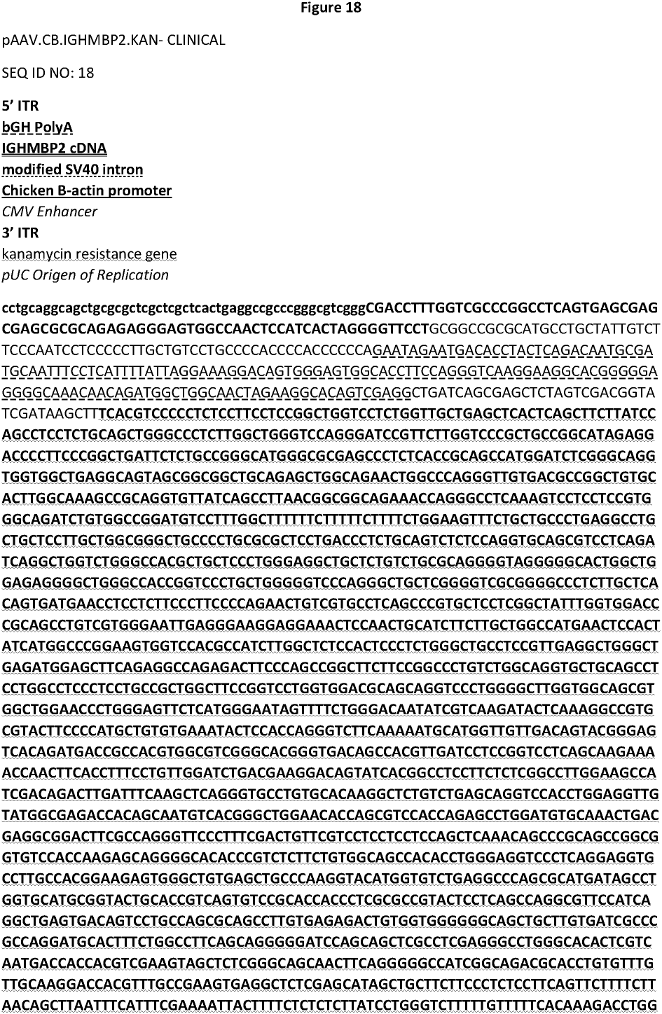

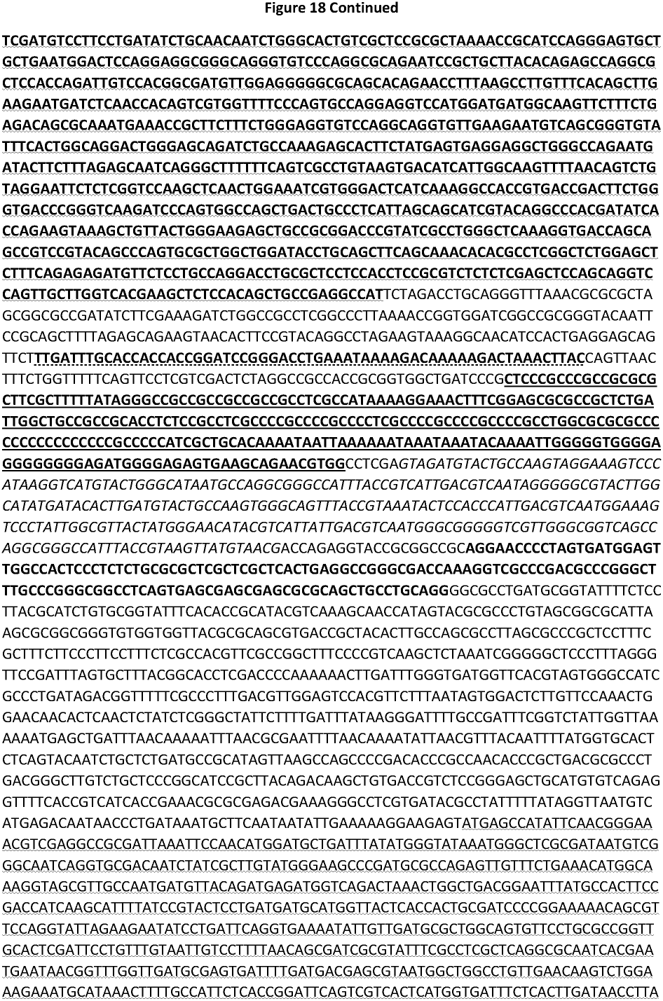

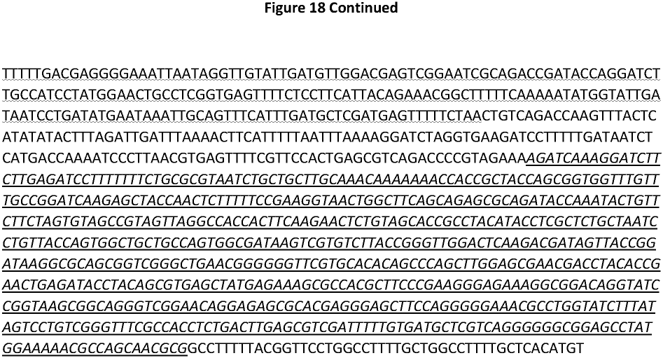

The map for plasmid ssAAV.CB.IGHMBP2.Kan-clinical (the CMV enhancer sequence, CB promoter sequence, IGHMBP2 cDNA sequence, the SV40 intron and the bGH polyadenylation sequence are in reverse orientation) is set out in FIG. 17 and the sequence of the entire plasmid is provided in SEQ ID NO: 18. The ssAAV.CB.IGHMBP2-clinical vector comprises the nucleotide sequence within and inclusive of the ITR’s, and as shown in FIG. 18. The rAAV vector comprises the 5′ AAV2 ITR (SEQ ID NO: 19), CMV enhancer, CB promoter, a modified SV40 intron sequence, the coding sequence for the IGHMBP2 gene, bGH polyA, and 3′ AAV2 ITR (SEQ ID NO: 12). The plasmid set forth in SEQ ID NO: 18 further comprises kanamycin resistance with pUC origin of replication. The kanamycin resistance gene is in the forward orientation in FIG. 17, but plasmids with the kanamycin resistance gene in the reverse orientation are also contemplated.

Table 5 shows the molecular features of the plasmid ssAAV.CB.IGHMBP2.Kan-clinical (SEQ ID NO: 18), in which range refers to the nucleotides in SEQ ID NO: 18 and ► indicates the element is in the forward orientation and the ◄ indicates the element is in the reverse orientation.

TABLE 5

| Type | Name | Range | Strand | Length | Description |
|---|---|---|---|---|---|
| repeat_reg!on | AAV2 ITR | 4246..4386 | ► | 141 | inverted terminal repeat of adeno-associated virus serotype 2 |
| misc_feature | AAV2 ITR | 1..130 | ► | 130 | AAV2 ITR |
| misc_feature | CMV Enhancer | Complement (3946..4225) | ◄ | 280 | CMV Enhancer |
| misc_feature | Chicken B-actin promoter | Complement (3670..3939) | ◄ | 270 | Chicken B-actin promoter |
| misc_feature | IGHMBP2 cDNA | Complement (385..3366) | ◄ | 2982 | IGHMBP2 cDNA |
| CDS | KanR | 5198..6007 | ► | 810 | aminoglycoside phosphotransferase |
| rep_origin | Ori | 6162..6781 | ► | 620 | high-copy-number ColE1/pMB1/pBR322/pUC origin of replication |
| misc_feature | bGH PolyA | Complement (200..346) | ◄ | 147 | bGH PolyA |
| misc_feature | modified SV40 intron | Complement (3545..3604) | ◄ | 60 | modified SV40 intron |

### Example 2 - CSF Delivery of IGHMBP2 Gene Therapy Vectors in Mice

Mouse models of SMARD1 and CMT2S were used to compare the effect of CSF delivery of AAV expressing IGHMBP2. Table 4 below provides different mouse model that may be used to investigate the efficacy of the IGHMBP2 gene therapy vectors. In this study, two different mouse models representing the very severe end of the disease spectrum (em3) and an intermediate disease form (nmd-2J) were used for this study.

TABLE 6

| | | | |
|---|---|---|---|
| Ighmbp2 mouse allele | Consequence | Homozygous Phenotype | Work Performed |
| nmd-2J | Splicing, reduced WT | Intermediate SMARD 1-like, paralysis, Dies -60-100 Days | Corti: Survival, strength, motor neuron and axon counts, muscle fiber size; Amold/Meyer: Survival, nerve conduction, bio distribution |
| em3 (nmd-J) | L362del | Severe SMARD1-like, paralysis, Dies -21 Days | Cox et al. (Neuron 21: 1327-1337, 1998) Survival, nerve conduction, strength, axon counts |
| Em5 | Y918C (CMT2S patient allele - Y920C) | CMT2S, sensory and motor neuropathy, Lives >10 Months | Cox: Survival, nerve conduction, strength, axon counts |

### Em3 Mouse Model

For the initial study, nmd^{em3/em3} homozygote mice were administered IGHMBP2 gene therapy vectors or controls via intracerebroventricular injection (ICV) into the cerebrospinal fluid (CSF). The ‘nmd’ mouse mutation causes progressive degeneration of spinal motor neurons and muscle atrophy (Cox et al., euron 21: 1327-1337, 1998). The nmd mutation was identified as the putative transcriptional activator and ATPase/DNA helicase previously described as Smbp2 or Catfl. The nmd phenotype is attenuated in a semidominant fashion by a major genetic locus on mouse chromosome 13.

Nmd^{em3/em3} homozygote mice mice received one intracerebroventricular injection of either of 5e10 viral genomes (vg) per animal of either ssAAV9.CB.IGHMBP2 (Virus A) or ssAAV9.P546.IGHMBP2 (Virus C) or empty viral particles (Virus B) formulated in 1x PBS and 0.001% Pluronic F68 (denoted as PBS/F68). Strength testing was performed starting from 3 weeks post days after administration, and survival was also monitored. FIG. 3 demonstrates that the treatment with Virus A and Virus C appeared to rescue paralysis but these mice remained smaller than controls.

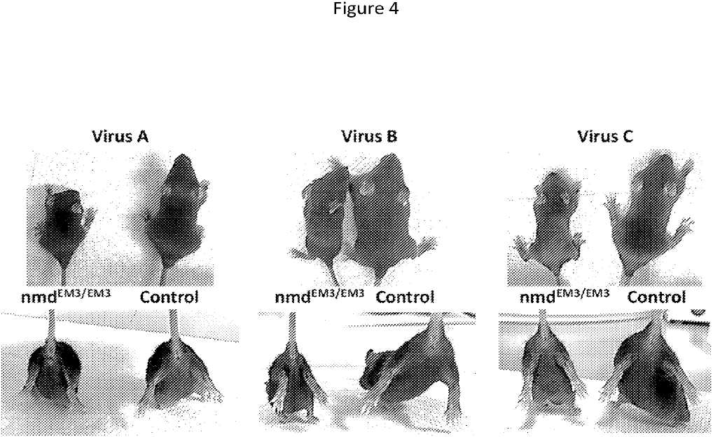

FIG. 4 provides representative images of nmd^{em3/em3} homozygote mice next to control mice at 2-3 weeks post-administration of AAV. The mice treated with Virus A or Virus C exhibited improved clasping. In addition, the mice treated with Virus B (empty viral particles) were unable to spread their hindlegs compared to the mice treated with Virus A and C.

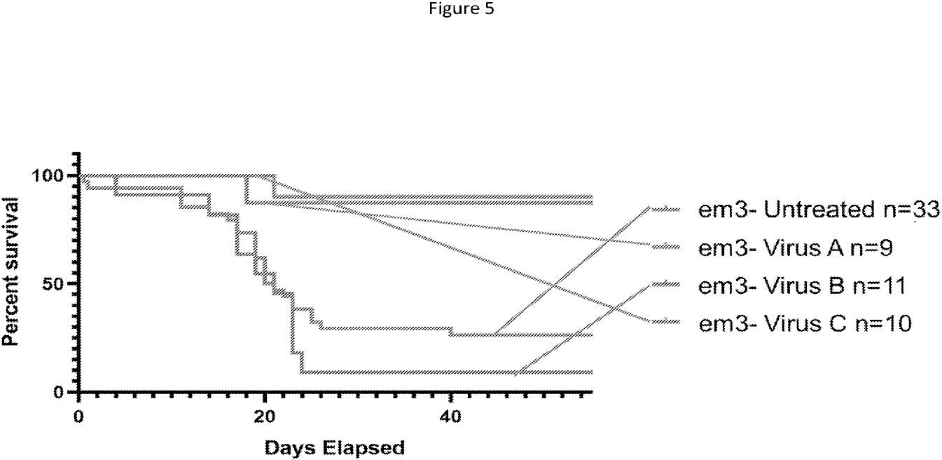

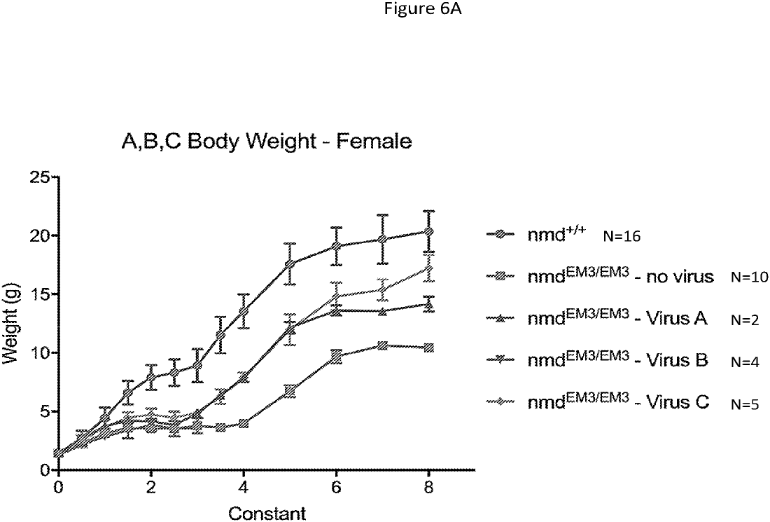

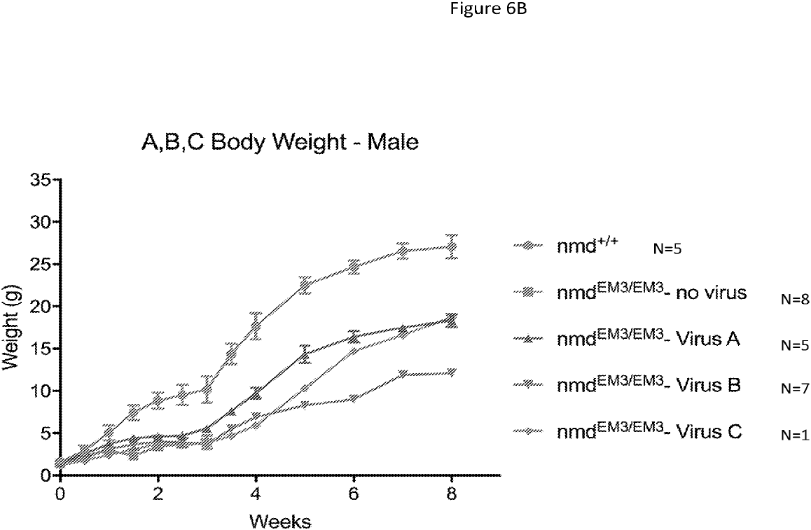

FIG. 5 provides the survival analysis of the nmd^{em3/em3} mice. Treatment with Virus A or Virus C rescued the lifespan of the mice (1 died early each). However, Virus B (empty viral particles) does not appear to rescue. There were the occasional survivors in non-treated and virus B treated, but these mice were very small and weak. As shown in FIG. 6, treatment with Virus A or Virus C improved body weight of the nmd^{em3/em3} homozygote mice but did not rescue the body weight completely as measured 8 weeks after treatment.

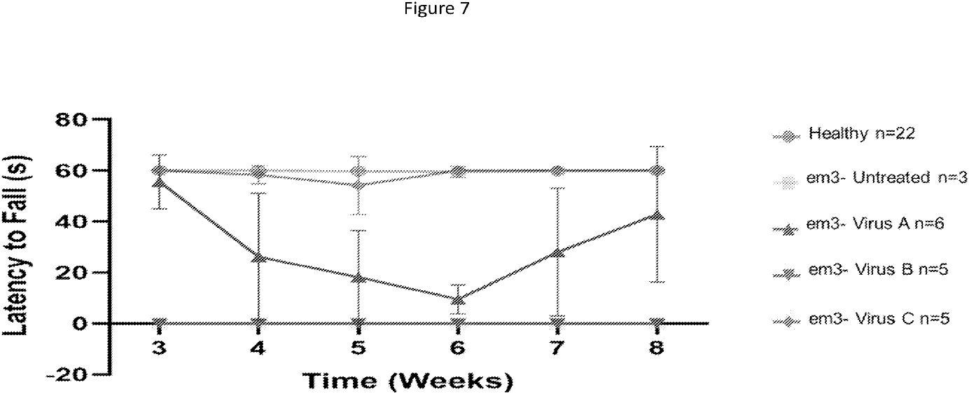

For strength testing, the treated mice were inverted on a wire cage lid for up to 60 seconds, and time was noted when they fell off or reach the 60-second endpoint. The longest time of three trials was recorded. If the mice failed their first attempt at holding 60 seconds, they were tested again to see if the first failed attempt was from: A) willfully not wanting to participate, B) slipped, or C) not being able to complete the 60 second wire hang. If they failed a second time, they were given ~ 3-5-minute break and tested again. The numbers in data/graph in FIG. 7 reflect the longest attempts. FIG. 7 provides the time after treatment verse the latency to fall as the mouse hangs from a wire. Treatment with Virus A showed an initial improvement, followed by decline of strength and later rescue back to wild type levels at 8 weeks post injection. Treatment with Virus C improved strength so that the homozygotic mice were similar to the normal nice (nmd^{+/+}).

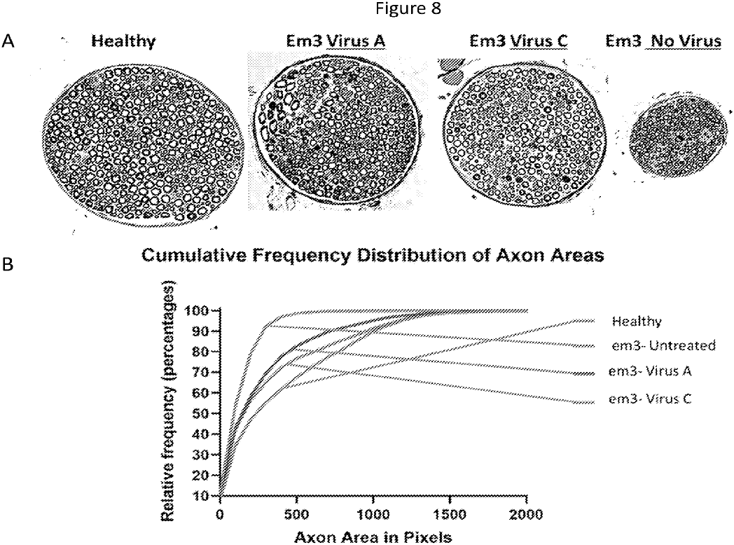

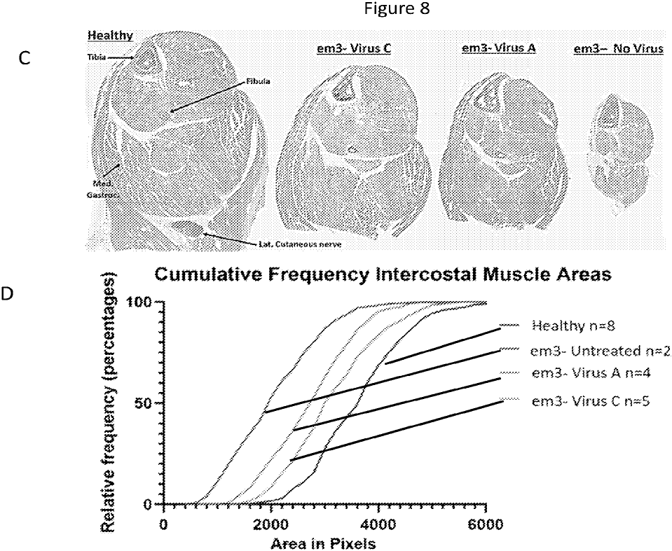

In addition, the nerves of the treated nmd^{em3/em3} homozygote mice were extracted and evaluated. Both left and right phrenic nerves, femoral motor nerves, and femoral sensory nerves were extracted and fixed in electron microscopy (EM) Fix overnight. The nerves were then washed 3x with 1x PBS buffer and stored at 4° C. until they are sent to our Histology core. Axon counts were determined using a semi-automated “WEKA Trainable Program” plug-in in ImageJ. The cross sections of the femoral nerve of the treated nmd^{em3/em3} homozygote mice are shown in FIG. 8A, and the axon area is provided in FIG. 8B. All groups significantly differed from each other with Kolmogorov-Smirnov test. The treatment with Virus A or Virus C significantly increased the area of the axons.

The muscle area of the intercostal muscle of the treated nmd ^{em3/em3} homozygote mice 8 weeks after treatment was also investigated. In the treated mice, 300 muscle fibers were manually traced in ImageJ from each animal’s hematoxylin & eosin cross section of the respective tissue. The measurements were provided in pixels and the graph in FIG. 8D represents cumulative frequency showing the range of muscle fibers areas.

The cross-section of the hind limb muscles are shown in FIG. 8C. For cross sectional images from hind limbs, two cuts were made on the patella and calcaneum to define length of leg at knee and ankle. A third cut was made at an equidistant point from patella and calcaneum. Bone and muscle morphology were examined to pick images from the same area between all animals. Mice treated with virus A or virus C showed increased muscle area, with virus C showing the best performance.

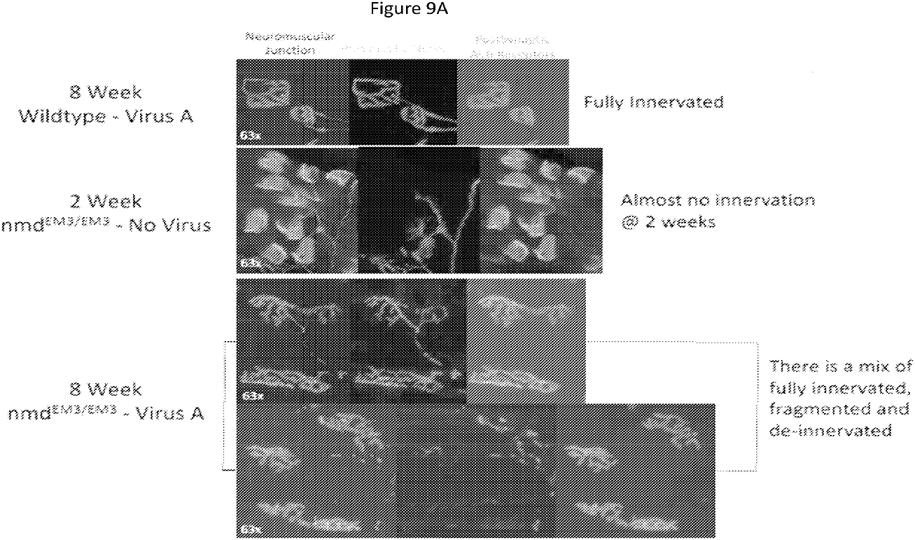

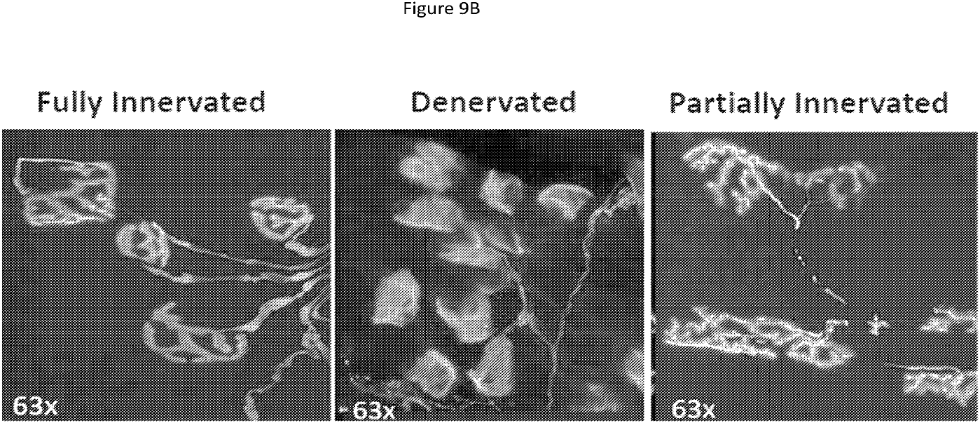

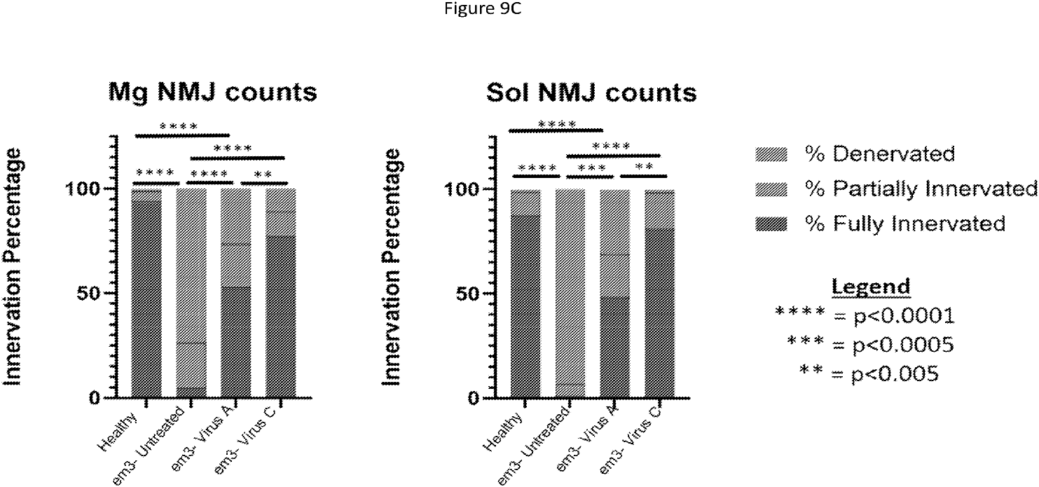

In addition, treatment with Virus A or Virus B partially rescued the neuromuscular junctions in the hind limbs of nmd^{em3/em3} homozygote mice. FIGS. 9A and 9B are representative photos showing immunocytochemistry of MG and Sol muscle obtained from the hind limb 8 weeks after treatment with rAAV. The MG and Sol muscle were weighed, the muscles were fixed with 4% PFA. The muscle was stained with an antibody detecting neurofilament (green) as a marker for presynaptic nerves and bungarotoxin (red) as a marker for the post synaptic Ach receptors. Neuromuscular junctions (NMJs) were photographed via SP8 Leica Confocal Microscope. Examples of each type of NMJ occupancy have been provided in FIG. 9A. Occupancy was determined manually as either fully innervated (red BTX acetylcholine channels and green neurofilament channel completely overlapping), partially innervated (red and green channels partially overlapping), or completely denervated (one channel being completely absent). There was almost no innervation of the neuromuscular junction in the hind limbs of untreated nmd^{em3/em3} homozygote mice. In the Virus A and C treated nmd^{em3/em3} homozygote mice, a mix of fully innervated, fragmented and non-innervated NMJs were found. The graph provides counts of NMJ on medial gastrocnemius and soleus, showing an increase of fully innervated NMJ with both Virus A and Virus C, with Virus C showing a higher % of fully innervated NMJs.

### Nmd-2J Mouse Model

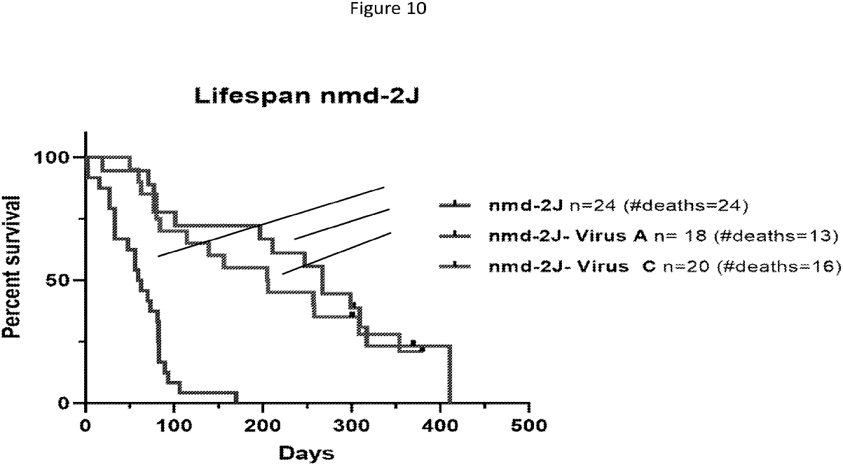

In addition, nmd-2J mice were administered IGHMBP2 gene therapy vectors or controls were administered via intracerebroventricular injection (ICV) into the cerebrospinal fluid (CSF). The nmd-2J mice have an intermediate SMARD1-like paralysis, and tend to die at about 60-100 days. These mice received one intracerebroventricular injection of either of 5e10 viral genomes (vg) per animal of either ssAAV9.CB.IGHMBP2 (Virus A) or ssAAV9.P546.IGHMBP2 (Virus C) or empty viral particles (Virus B) formulated in 1x PBS and 0.001% Pluronic F68 (denoted as PBS/F68). As described above, FIG. 10 provides a survival plot that demonstrates Virus A and C improved survival nmd-2J mice.

Electromyography (EMG) is a potential clinical biomarker for efficacy. Electrophysiological outcomes include compound muscle action potential (CMAP), single motor unit potential (SMUP) and motor unit number estimation (MUNE) recorded from the hind limb muscles following sciatic nerve stimulation as described in Arnold et al, Annals of clinical and translational neurology, 1(1), 34-44, 2014. CMAP measures strength of innervation, and MUNE provides an estimate of the number of neurons innervating a muscle.

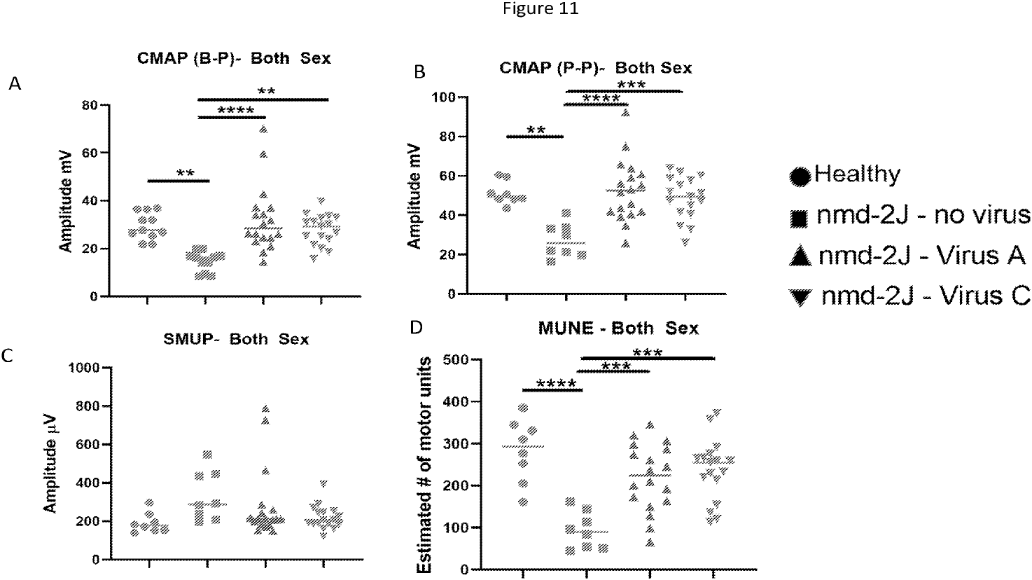

Briefly, two fine ring electrodes were placed on anesthetized mice for recording the electrophysiological outcomes. The active (E1) ring electrode was placed on the skin over the proximal portion of the gastrocnemius muscle of the hind limb, at the knee joint, and the reference (E2) ring electrode was placed over the region of mid-metatarsal portion of the foot. The skin underlying the ring electrodes was coated with gel to reduce impedance. Sciatic CMAP responses were obtained with supramaximal stimulation (~120% maximal) of the sciatic nerve (square-wave pulses of 0.1 ms duration and intensity: 1-10 mA). Average Single Motor Unit Potential (SMUP) Size and MUNE were calculated by recording incremental responses by delivering submaximal stimulation of 0.1 ms duration at a frequency of 1 Hz while increasing the intensity in 0.026 mA steps to obtain the minimal all-or-none responses. Ten increments were averaged to provide an average single motor unit potential SMUP amplitude. MUNE was calculated as follows: MUNE=CMAP/average SMUP. For CMAP and SMUP amplitudes peak-to-peak measurements were used. As shown in FIGS. 11A-D, there was a significant increase on CMAP and MUNE in mice treated with Virus A or Virus C, but the CMAP was not different in mice treated with Virus A and mice treated with Virus C. The data provide in FIGS. 11A-D demonstrates a clear difference between mice treated with IGHMBP2 gene therapy vectors as compared to untreated mice.

### Em5 Mouse Model

The em5 mouse model was also used to investigate the effect of IGHMBP2 gene therapy vectors administered via intracerebroventricular injection (ICV) into the cerebrospinal fluid (CSF). The em5 mice have CMT2S, sensory and motor neuropathy phenotype and have a lifespan of about 10 months. These mice received one intracerebroventricular injection of either of 5e10 viral genomes (vg) per animal of either ssAAV9.CB.IGHMBP2 (Virus A) or ssAAV9.P546.IGHMBP2 (Virus C) or empty viral particles (Virus B) formulated in 1x PBS and 0.001% Pluronic F68 (denoted as PBS/F68).

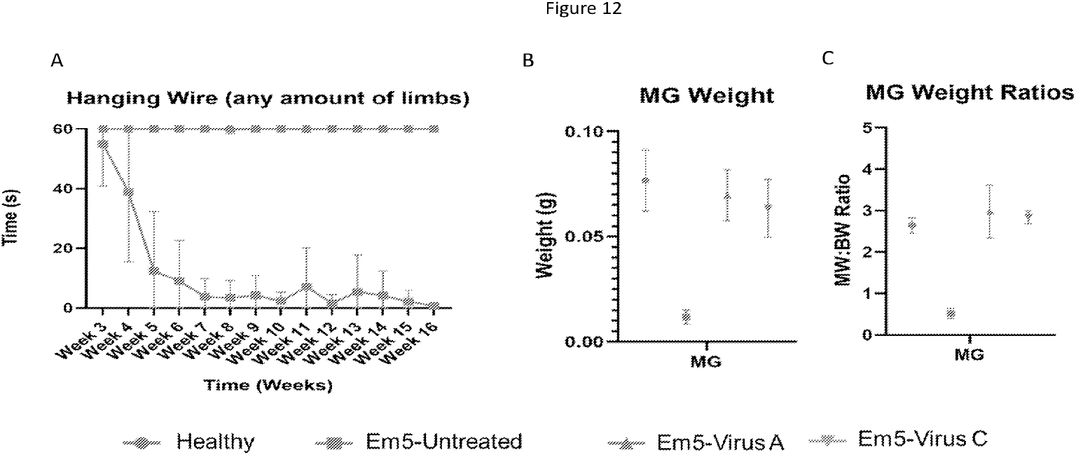

FIG. 12A provides results from a hanging wire test on healthy mice and on Em5 mice treated with Virus A and Virus C. The healthy mice and the mice treated with Virus A and Virus C showed significantly better grip strength compared to untreated em5 mice. There was no significant difference between the healthy mice and the mice treated with Virus A or Virus C. FIG. 12B shows the weight of the medial gastrocnemius (MG) in the treated and untreated mice. FIG. 12B shows an increase in muscle mass of the MG in relation to total body weight in healthy and Virus A and Virus C treated Em5 mice as compared to the untreated em5 mice. There was no difference between treated and healthy animals.

### Example 3 - Clinical Trial in Humans

The ssAAV9.CB.IGHMBP2 (Virus A) or ssAAV9.P546.IGHMBP2 (Virus C) is delivered intrathecally to human patients suffering from an IGHMBP2-related disorder, such as SMARD1 or CMT2S. The scAAV for the clinical trial is produced utilizing a triple-transfection method of HEK293 cells, under cGMP conditions.

Patients selected for participation will be 1-20 years of age with a diagnosis of an IGHMBP2-related disorder, such as SMARD1 or CMT2S as determined by genotype. The patients receive a one-time gene transfer dose of ssAAV per patient. The ssAAV is formulated in 20 mM 1 mM MgCl₂, 200 mM NaCl, 0.001% poloxamer 188 Tris (pH8.0), and will be delivered one-time through an intrathecal injection. Safety is assessed on clinical grounds, and by examination of safety labels. There is a minimum of 3-4 weeks between enrollments of each subject to allow for review of Day 30 post-gene transfer safety data. Disease progression is measured and the impact of treatment on quality of life and potential for prolonged survival is assessed.

1. A polynucleotide comprising
(a) one or more regulatory control elements; and
(b) an immunoglobulin-µ binding protein 2 (IGHMBP2) cDNA sequence.

2. The polynucleotide of claim 1, wherein the regulatory control element is CBA promoter or P546 promoter, or fragments thereof.

3. The polynucleotide of claim 1, wherein the IGHMBP2 cDNA comprises the polynucleotide sequence comprising at least 95% sequence identity to SEQ ID NO: 1 or the nucleotide sequence set forth in SEQ ID NO: 1.

4. The polynucleotide of claim 1 comprising the nucleotide sequence of SEQ ID NO: 3 or 4.

5. A recombinant adeno-associated virus (rAAV) having a genome comprising a polynucleotide sequence of claim 1.

6. (canceled)

7. (canceled)

8. The rAAV of claim 5, wherein the rAAV is of the serotype AAV1, AAV2, AAV3, AAV4, AAV5, AAV6, AAV7, AAV8, AAV9, AAVRH10, AAVRH74, AAV11, AAV12, AAV13 or Anc80, AAV7m8 and their derivatives.

9. An rAAV particle comprising the rAAV of claim 5 .

10. A composition comprising the rAAV of claim 5 .

11. The composition of claim 10 further comprising an agent that increases the viscosity or density of the composition.

12. (canceled)

13. The composition of claim 9, wherein the composition is formulated for direct injection into the cerebrospinal fluid, intracerebroventricular delivery, intrathecal delivery or intravenous delivery.

14. The composition of claim 10, wherein the composition is formulated for intrathecal delivery and comprises a dose of rAAV or rAAV particles of about 1e13 vg per patient to about 1e15 vg per patient or the composition is formulated for intravenous delivery and comprises a dose of rAAV or rAAV particles of about 1e13 vg/kg to about 2e14 vg/kg.

15. The composition of claim 10, wherein the composition is formulated for intravenous delivery and comprises a dose of rAAV or rAAV particles of about 1e13 vg/kg to about 2e14 vg/kg.

16. A method of treating an IGHMBP2 -related disorder in a subject in need thereof comprising administering an rAAV of claim 5.

17. The method of claim 16, wherein the disorder is SMARD1 or CMT2S.

18. The method of claim 16 , wherein the subject has a mutation in the IGHMBP2 gene.

19. The method of claim 16, wherein the rAAV are administered by direct injection into the cerebrospinal fluid, intracerebroventricular delivery, intrathecal delivery or intravenous delivery.

20. The method of claim 16, wherein a dose of rAAV of about 1e13 vg per patient to about 1e15 vg per patient is administered by intrathecal delivery to the subject.

21. The method of claim 16, wherein a dose of rAAV or rAAV particles of a dose of about 1e13 vg/kg to about 2e14 vg/kg is administered by intravenous delivery to the subject.

22. The method of claim 16, further comprising a step of administering an immunosuppressing agent.

23-33. (canceled)

34. A composition comprising the viral particle of claim 9.
